(19) United States
(12) Patent Application Publication
(10) Pub. No.: US 2021/0307963 A1
(43) Pub. Date: Oct. 7, 2021

(54) Active Agent Delivery Devices and Methods for Using the Same

(71) Applicant: iRenix Medical, Inc., Palo Alto, CA (US)

(72) Inventors: Stephen J. Smith, Palo Alto, CA (US); Thomas W. Chalberg, Jr., Palo Alto, CA (US); Espir Gabriel Kahatt, Palo Alto, CA (US); Greg Yedinak, Palo Alto, CA (US)

(21) Appl. No.: 17/269,348

(22) PCT Filed: Aug. 21, 2019
(86) PCT No.: PCT/US2019/047428

§ 371 Date: Feb. 18, 2021

Related U.S. Application Data
(60) Provisional application No. 62/722,447, filed on Aug. 24, 2018.
(60) Provisional application No. 62/722,454, filed on Aug. 24, 2018.
(60) Provisional application No. 62/722,657, filed on Aug. 24, 2018.

(51) Int. Cl. A61F 9/00; A61M 5/315; A61M 5/32; A61M 5/20

(52) U.S. Cl. CPC A61F 9/0008; A61M 5/31533; A61M 5/32; A61M 2005/3125; A61M 2202/048; A61M 2210/0612; A61M 5/20

(57) ABSTRACT

Therapeutic agent delivery devices are provided. Aspects of the devices include an active agent composition and an actuator configured to deliver the active agent composition to a target delivery site. In some instances, aspects of the devices include a syringe comprising a viscous active agent composition, a needle operably coupled to the syringe and an actuator configured to deliver viscous active agent composition from the syringe through the needed to a target delivery site, such as an intravitreal site. In some instances, aspects of the devices include a sleeve comprising a solid formulation of the active agent; a needle operably coupled to the sleeve; and an actuator configured to move the solid formulation from the sleeve and through the needle, e.g., to an intravitreal location. In some instances, aspects of the devices include a syringe containing a composition for subcutaneous delivery, a needle operably coupled to the syringe, a motorized actuator configured to deliver the composition from the syringe through the needle to a target subcutaneous site, and a pain mitigation system. Also provided are methods of using the devices.

### CROSS-REFERENCE TO RELATED APPLICATIONS

Pursuant to 35 U.S.C. § 119 (e), this application claims priority to the filing date of the U.S. Provisional Patent Application Ser. No. 62/722,447, filed Aug. 24, 2018; U.S. Provisional Patent Application Ser. No. 62/722,454, filed Aug. 24, 2018; and U.S. Provisional Patent Application Ser. No. 62/722,657, filed Aug. 24, 2018; the disclosure of which applications are herein incorporated by reference.

### GOVERNMENT RIGHTS

This invention was made with Government support under contract R44 EY028495 awarded by the National Institutes of Health. The Government has certain rights in the invention.

### INTRODUCTION

Pain is a major limiting factor in many common procedures performed in the inpatient and ambulatory care settings. A very abbreviated list of such procedures includes skin biopsy, fine needle aspiration biopsy, IV insertion, vaccination, injections (including injection of anesthetics and gasses), blood draws, central line placements, and finger and heal pricks for blood analysis (glucose measurement). Pharmacologic anesthesia is a primary method of pain reduction, but the delivery of local pharmacologic anesthesia usually requires a painful injection.

The ocular surface is a tissue surface to which therapeutic agents may be delivered. The ability to deliver medication directly into the eye via intravitreal injection therapy (IVT) has transformed the treatment landscape of a number of previously blinding diseases, including macular degeneration and diabetic retinopathy. The success of these therapies in preventing blindness has resulted in a dramatic increase in the number of intravitreal injections performed, with an estimated 4.1 million injections given in the United States alone in 2013. The number of indications for IVT continues to expand, increasing utilization of this therapy significantly every year. The primary limitations of IVT are patient discomfort, ocular surface bleeding, corneal toxicity, and the time constraints of treating the vast number of patients requiring this therapy. These drawbacks relate to the difficulty of delivering ocular anesthesia to the highly vascularized ocular surface.

To give an ocular injection, the physician first provides ocular surface anesthesia by one or more of a number of methods, including the following: topical application of anesthetic drops; a subconjunctival injection of lidocaine; placement of cotton tipped applicators (commonly called a “pledget”) soaked in lidocaine over the planned injection site, application of topical anesthetic gel, or some combination of these. Following ocular anesthesia, the physician or an assistant sterilizes the periocular region by coating it in betadine or a similar antiseptic. Optionally, an eyelid speculum is placed, and the physician marks the location of the injection using calipers that guide placement of the needle. The ocular surface is again sterilized, and the physician gives the injection. Current methods of local anesthesia have unique drawbacks and patients often experience discomfort during and after intraocular injections.

### SUMMARY

Therapeutic agent delivery devices are provided. Aspects of the devices include an active agent composition and an actuator configured to deliver the active agent composition to a target delivery site. In some instances, aspects of the devices include a syringe comprising a viscous active agent composition, a needle operably coupled to the syringe and an actuator configured to deliver viscous active agent composition from the syringe through the needed to a target delivery site, such as an intravitreal site. In some instances, aspects of the devices include a sleeve comprising a solid formulation of the active agent; a needle operably coupled to the sleeve; and an actuator configured to move the solid formulation from the sleeve and through the needle, e.g., to an intravitreal location. In some instances, aspects of the devices include a syringe containing a composition for subcutaneous delivery, a needle operably coupled to the syringe, a motorized actuator configured to deliver the composition from the syringe through the needle to a target subcutaneous site, and a pain mitigation system. In some instances, aspects of the devices include a syringe containing a composition for injection into a drug reservoir, such as an on-body pump or ocular drug depot system. Also provided are methods of using the devices.

Some embodiments of the invention include portable, handheld devices with an electronic motor calibrated to deliver a viscous drug composition, such as viscous drug composition of a complement inhibitor, to the eye with a range of volumes from 5-150 μl. The motorized actuator, e.g., electronic motorized actuator, may be capable of delivering a viscous substance through a small gauge needle. The hand-held device may include a gripping portion for ease of use. In some instances, the device includes a syringe that clips into the device, such as a sterilized or non-sterilized syringe. The syringe material may be polymeric, e.g., COP, COC, or glass. The devices can include the use of a single-use, sterile tip that shields the needle. In some instances, the tip, when present, does not include a metal foil, but may be of an inert plastic to serve as an injection guide (circular tip with opening 3.5 mm from the limbus). The viscosity of the delivered compositions may vary, ranging in some instances from 25-750 centipoise, such as from 100-500 centipoise. The devices may be configured for rapid delivery, such as 0.1 s to 60 seconds, including 0.5-10 seconds. The devices may be configured for precision of drug delivery measurement (electronic sensors guide delivery volume), and may include a mechanism to measure volume delivered. The devices may include a display, e.g., for showing drug name and volume to be delivered. The devices may include an alarm, e.g., for signaling completion of delivery, if drug delivery is aborted or incomplete, etc. The devices may be configured as multi-use devices or single-use devices, including where the motor and device body can be re-sterilized and “recyclable”. In some instances, the devices include tip vibration at the time of injection.

Yet other embodiments of the invention find use in the delivery of solid formulations of one or more active agents to a target delivery site, such as an ocular delivery site, e.g., an intravitreal site or an intracameral site. The solid formulations may be active agent depots that are configured to release one or more active agents over an extended period of time. The solid formulations may include one or more active agents, e.g., steroids, ROK inhibitors, etc., in a solid matrix, where the matrix may be polymeric, and may be biodegradable (i.e., bio-erodible). The solid formulation may be any convenience shape, such as a pellet, e.g., a rod-shaped pellet, or a shape-modifying substance (e.g., polymer that folds upon injection into a substance like vitreous, aqueous or cutaneous tissue). The sleeve(s) or syringe(s) may hold a single solid formulation or multiple distinct solid formulations, e.g., multiple pellets. The pellets may contain one drug or multiple drugs. The actuator may be motorized, spring loaded or manual, e.g., user advances a lever on the body of the device. The devices may be multi-use or single use devices, including where the motor and device body can be re-sterilized and “recyclable”. In some instances, a small, off-weight motor in the device may be present to cause vibration at the time of injection. The device may include a display, e.g., for confirmation of drug and dose. A monitoring mechanism internally in the sleeve may be provided which alerts user after the solid drug formulation has been injected. In some instances, a cooling base station, and one or more of the components of the device may be configured for communication, e.g., via wired or wireless protocols.

Yet other embodiments include cooling auto-injectors for the delivery of subcutaneous compositions, e.g., aesthetic compositions, such as botulinum toxin compositions, dermal fillers, etc., as well as other active agent compositions, e.g., immune modulators, glucose modulators, etc. In some instances, the devices include a tissue contacting tip, which may vary in size, as desired, and in some instances may be smaller, e.g., 0.5 mm², or larger. In some instances, a translucent tip material is provided to enable needle visualization throughout the injection. Programing may be provided that allows for one or more of: variable depth of the needle penetration, controlled rate of drug delivery, programable volume selection (e.g., where a desired volume of composition can be input into the device and the device correspondingly delivers the desired volume of composition, e.g., 0.1 ml, 0.05 ml, etc.), programable temperature selection. Needle retraction to protect patient safety may be provided. In some instances, the device includes features that enable patient use (single-use, retracting needle, alarms to verify device delivery). Where desired, a small, off-weight motor in the device that causes vibration at the time of injection may be provided. Devices may be configured to be employed with cooling base stations and/or provide for connectivity features.

### BRIEF DESCRIPTION OF THE FIGURES

FIG. 1 provides a view of a hand-held active agent delivery device for delivering a viscous composition according to an embodiment of the invention.

FIG. 2 provides a view of a hand-held solid formulation active agent delivery device according to an embodiment of the invention.

FIG. 3 provides a view of a hand-held active agent subcutaneous delivery device according to an embodiment of the invention.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular conjunctival tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

The term “sterile” is used in conventional sense to denote free from live bacteria or other microorganisms. A “sterile field” is an area within the operating theater/clinic within which only sterile equipment can be used, and into which only those personnel who have gone through surgical scrubbing and the gowning process can enter.

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Modules are made up of one or more functional blocks which act in concert to perform a particular function, which is the purpose of the module. A given module may be implemented as hardware, software or a combination thereof. In some instances, modules may include a circuitry element, such as an integrated circuit. When present, integrated circuits may include a number of distinct functional blocks, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Furthermore, the definitions and descriptions provided in one or more (e.g., one, two, three, or four, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Therapeutic agent delivery devices are provided. Aspects of the devices include an active agent composition and an actuator configured to deliver the active agent composition to a target delivery site. In some instances, aspects of the devices include a syringe comprising a viscous active agent composition, a needle operably coupled to the syringe and an actuator configured to deliver viscous active agent composition from the syringe through the needed to a target delivery site, such as an intravitreal site. In some instances, aspects of the devices include a sleeve comprising a solid formulation of the active agent; a needle operably coupled to the sleeve; and an actuator configured to move the solid formulation from the sleeve and through the needle, e.g., to an intravitreal location. In some instances, aspects of the devices include a syringe containing a composition for subcutaneous delivery, a needle operably coupled to the syringe, a motorized actuator configured to deliver the composition from the syringe through the needle to a target subcutaneous site, and a pain mitigation system. Also provided are methods of using the devices.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

### Viscous Active Agent Delivery Devices

As summarized above, active agent delivery devices are provided. As the devices are active agent delivery devices, they are configured to deliver an amount (e.g., dosage) of an active, e.g., therapeutic, agent to a target location, e.g., via a target delivery site, of a subject. As will be reviewed in greater detail below, in some embodiments the active agent is present in a viscous composition. While the dosage of viscous active agent composition that is delivered to a target tissue by the devices may vary, in some instances the dosage ranges from 5 μl to 150 μl, such as from 10 μl to 100 μl and including 10 μl to 50 μl.

The term “syringe” is used in its conventional sense to refer to a device for injecting fluids. In some instances, a syringe includes a body, such as a tubular body, e.g., a calibrated cylindrical barrel (which may be fabricated from glass or plastic), having a plunger at a first or proximal end and an orifice at a second or distal end, e.g., for providing passage of the active agent composition from the inside of the syringe into a needle which may be operably engaged to the distal end of the container. The syringe is configured to hold a desired amount of an active agent composition. While the volume of the syringe may vary, in some instances the volume ranges from 0.1 to 5.0 ml, such as 0.1 to 1.50 ml, including 0.1 to 1.0 ml, e.g., 0.70 to 0.80 ml. In some instances, the volume of the syringe is sufficient to hold an amount of an active agent composition that is greater than the amount which is delivered to a target tissue delivery site during use of the device. While the magnitude of the excess may vary, in some instances the magnitude ranges from 110% to 500% of the delivered volume, such as 120% to 150%. Where the active agent composition is a non-gaseous composition, the amount of any gas, e.g., air, in the reservoir (and other components of the active agent delivery system) may be minimal, where in some instances the amount is 10 μl or less, such as 5 μl or less, 3 μl or less, 2 μl or less, or 1 μl or less. As indicated above, while the dosage of liquid active agent composition that is delivered to a target tissue by the devices may vary, in some instances the dosage ranges from 5 μl to 100 μl, such as from 10 μl to 50 μl. In some instances, the syringe may include a dosing mark, e.g., an optically recognizable identifier that indicates delivery of a dosage of active agent composition. The dosage that is indicated by the dosing mark may vary, and, in some instances, ranges from 10 to 100 μl, such as 50 μl. The syringe may be fabricated from any convenient material, including glasses (e.g., type 1 class), plastics (such that the container is a polymeric container, e.g., cyclic olefin polymer (COP) and cyclic olefin copolymer (COC)), etc. Suitable materials include, but are not limited to, those described in published PCT application publication nos. WO 2013/178771; WO2015/173260; WO2017/087798 and WO2017/085253; the disclosures of which are herein incorporated by reference.

In some embodiments, the syringe is not surface sterilized. As the syringe is not surface sterilized, the outer surface of the syringe is non-sterile. Non-sterile means any that must be considered to be not sterile or cannot be considered to be sterile, e.g., because of the history of the surface, such as the handling/processing history of the surface (for example as determined by standard medical procedures). In some instances, the non-sterile surface is one that has not been treated so that it is free of live bacteria or other microorganisms (for example, the surface has not undergone a sterilization procedure, such as a heat sterilization procedure (e.g., steam exposure) or chemical sterilization procedure, (e.g., exposure to ethylene oxide gas, exposure to hydrogen peroxide gas plasma, peracetic acid immersion, ozone exposure, etc.). In some instances, the non-sterilized surface has at least one live bacteria or other microorganism present thereon. While the outer surface of the syringe is not sterile, the interior of the syringe, as well as the contents thereof, e.g., liquid active agent composition, are sterile.

Any desired active agent composition may be present in a container (i.e., reservoir) of the syringe, where an active agent composition may include a single active agent or combination of two or more difference active agents, as desired. An active agent is any component that provides pharmacological activity or other direct effect in the diagnosis, cure, mitigation, treatment, or prevention of disease, or affects the structure or any function of the body of man or animals, and may be a liquid, gel, or solid. A type of active agent that may be present in a given active agent composition is a therapeutic agent, which is an agent that may be used in treating, remediating, or curing a disorder or disease. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids, such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; antibody biopolymer conjugates, drug eluting microparticles such as poly-lactide-co-glycolide acid (PLGA), aminosterols such as squalamine; antihistamines such as H 1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; glucose modulators, e.g., insulin; immune modulators, etc.

The active agent or agents are present in the active agent composition in a therapeutically effective amount. The amount of a given active in an active agent composition may vary, e.g., depending on the particular active agent, target condition, etc., where in some instances the amount of a given active agent ranges from 0.0005 to 10000 mg/ml, such as 10 to 500 mg/ml and including 25 to 200 mg/ml.

In addition, the active agent or agents, a given active agent composition include pharmaceutically acceptable delivery vehicle, e.g., a pharmaceutically acceptable aqueous vehicle. For pharmaceutically acceptable aqueous vehicles, in addition to water, the aqueous delivery vehicle may include a number of different components, including but not limited to: salts, buffers, preservatives, solubility enhancers, viscosity modulators, colorants, etc. Suitable aqueous vehicles include sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, viscosity modifiers, emulsifiers and stabilizers, antimicrobial agents, pH adjusting agents. The viscosity of a given active agent composition may vary. In some instances, the viscosity ranges from 25 to 1500 centipoise, such as 50 to 750 centipoise, and including 100 to 500 centipoise.

A given syringe may include in the container or reservoir thereof a single active agent composition or two or more distinct active agent compositions. The two or more active agent compositions may differ from each other in terms of active agent(s), delivery vehicle, viscosity, etc. Where a given syringe includes two or more distinct active agent compositions, the distinct active agent composition may be separated from each other by a barrier, such as a frangible barrier, such that each distinct active agent composition is present in its own chamber of the syringe. In such instances, the syringe may have two or more chambers, e.g., one for each distinct active agent composition container therein. During delivery, the barrier(s) may be disrupted to provide for combination of the active agent compositions. For example, where two chambers are separated from each other by a frangible barrier, movement of the plunger during active agent composition delivery may disrupt the frangible barrier, allowing for mixing of the two or more active agent compositions. Where desired, a barrier disrupting mechanism may be employed, e.g., a barrier piercing mechanism, etc.

A given device may include a single syringe filed with one or more active agent compositions, e.g., as described above, or two or more syringes each filled with one or more active agent compositions, such as a device that includes two syringes each filled with a different active agent composition. Where a device includes two or more syringes each filled with a different active agent composition, the differing active agent compositions may differ from each other in terms of active agent(s), delivery vehicle, viscosity, etc. In these embodiments, the devices may be configured to deliver the active agent compositions contained in the distinct syringes as separate compositions to the target tissue, or they may be configured to combine the active agent compositions of the disparate syringes prior to delivery to a target tissue. In embodiments where the devices are configured to deliver the active agent compositions contained in the distinct syringes as separate compositions to the target tissue, each syringe may be operatively coupled to its own needle during active agent composition delivery. Alternatively, each syringe may be operatively coupled to a single needle having a distinct internal lumen or passageway for the contents of each syringe, such as needle having a central divider that defines two passageways fluidically isolated from each other and operably coupled to a syringe of a dual syringe system. In embodiments where the devices are configured to combine the active agent compositions of the disparate syringes prior to delivery to a target tissue, the syringes may be operably coupled to single needle having a single passageway during delivery, such that the contents of the distinct syringes mix upon passage through the needle. Where desired, a mixing element may be provided in the device, e.g., at the interface of the syringes and the needle.

Where desired, devices of the invention may also include a tip having a sterile tissue contacting surface. In such embodiments, as the tissue contacting surface of the tip is sterile, as least the portion of the surface that contacts tissue during use of the device is free of live bacteria or other microorganisms. In some instances, the dimensions of the tissue contacting tip will be such that needle entry occurs at a predetermined distance from the corneal limbus (where distances may be as provided above) when the tissue contacting tip is placed on the ocular surface, where such configurations may negate any need for manual measurement for safe injection distance prior to procedure (intravitreal, intracameral etc.). As such, the tissue contacting tip may be configured to prevent contact with a tissue structure near a target tissue delivery site, e.g., an ocular lens or ocular retina where the target tissue delivery site is an ocular tissue delivery site. For example, the tissue contacting tip may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. While the tissue contacting surface may have any convenient configuration, in some instances the surface is planar. The shape of the tissue contacting surface may vary, where shapes of certain embodiments include circular, oval, triangular, rectangular, including square, hexagonal, octagonal, etc. The area of the tissue contacting surface may also vary, where in some instances the area ranges from 0.5 to 50 mm².

Devices of the invention also include a needle operably coupled to the syringe and, optionally, the tip when present. The needle is configured to convey the active agent composition from the syringe to a target tissue, e.g., via a target tissue delivery site. The needle may have any convenient dimensions, and, in some instances, has a gauge ranging from 20 to 35, such as 23 to 35, such as 27 to 35, e.g., 30 to 33, such as 29, 29½, 30, 31, 32, 33 and 34 gauge. The needle may include a single lumen or two or more separate lumens container within a common core, as desired, e.g., where the needle includes two separate lumens contained within a common core and has a gauge ranging from 23 to 30. The needle may be made of any convenient material, e.g., stainless steel, etc. At the proximal end of the needle may be a connector for operably connecting to the syringe. Any convenient connector may be present, where connector may be present, such as a luer connector, e.g., a luer slip or luer lock connector.

As summarized above, the needle is operably coupled to the syringe and, optionally, the tip. The proximal end of the needle may be operably engaged with the distal end of the syringe such that liquid composition inside of the syringe passes through the distal opening of the syringe and into the inside (i.e., bore) of the needle. The proximal end of the needle may be operably engaged with the distal end of the syringe using any convenient configuration. Examples of suitable configurations include, but are not limited to, press fit configurations, luer fitting configurations, e.g., luer slip or luer lock, etc.

In addition to being operably coupled to the syringe, when present the needle is also operably coupled to the tip that includes the sterile tissue contacting surface. In some instances, the tip is a structure having a proximal end attached to the needle, e.g., by a suitable fitting, and a distal end extending beyond the distal end of the needle, e.g., by a distance ranging from 2 to 50 mm, e.g., 2 to 20 mm, such as 2 mm to 10 mm, where the distal end includes the sterile tissue contacting surface. The tip may be made up of a single component or two or more components operably connected to each other. For example, a tip may include a first proximal component and a second distal component, where the distal component includes the sterile tissue contacting surface and the proximal component includes a fitting for operably coupling to the needle. The proximal component may further house the needle or a portion thereof, such that the proximal component may be considered to be a needle housing. The proximal and distal components may be configured to move relative to each other during use. The distance that the two components may move relative to each other may vary, where in some instances the distance ranges from 1 to 20 mm, such as 2 to 10 mm. In some instances, a locking mechanism maintains the distal end of the needle relative to the distal end of the tissue contacting tip prior to actuation. The locking mechanism may be releasable upon movement of the tissue contacting tip relative to the needle, such as a rotational movement, e.g., of the tissue contacting tip about the central longitudinal of the needle. For example, a tissue contacting tip may be turned, such as a quarter turn, relative to the associated needle which results in removing the lock and allowing the needle to be moved relative to the distal end of the tissue contacting tip. The distal, tissue contacting end of the tip may include an opening dimensioned to provide for passage of the distal end of the needle through the contacting tip during active agent delivery, thereby providing access of the needle directly to a target tissue. Upon actuation, the distal end of the needle moves relative to the distal end of the tissue contacting tip. The distance that the distal end of the needle extends beyond the distal end of the tissue contacting tip, and sterile surface thereof, during actuation and active agent delivery may vary, and, in some instances, ranges from 0.1 to 50 mm, such as 0.1 to 5.0 mm, including 0.5 to 4.0 mm. While the dimensions of the opening, when present, may vary, in some instances the opening has a diameter sufficient to accommodate passage of a needle having a gauge, e.g., as described above. The distal end of the tissue contacting tip may in some instances be fabricated from a thermally conductive material, e.g., a metal or alloy thereof, such as in those instances where the distal end of the tissue contacting tip operatively engages the distal end of a cooling element of a cooling system of the actuator component, e.g., as described in greater detail below. Other suitable materials may also be employed, such as polymeric materials. A given tip may be configured to be operably coupled to a single needle, or two or more needles. For example, a given tip may be configured to operably couple to two needles, e.g., where a device includes two syringes, such as described above.

In some instances, the tissue contacting tip may include one or more filters. The filters may be configured to remove particles or other unwanted components present in the active agent composition prior to delivery to the target tissue delivery site. Such filters may be configured to inhibit passage of particles above a certain pore size from >0.1 μm to >50 μm, such as >5 μm. The one or more filters may be positioned at any convenient location in the therapeutic agent delivery system, e.g., at the exit from the container into the injector, at some point along the injector, at the distal end of the injector, etc.

Where desired, the distal, tissue contacting surface may include a removable cover, e.g., that is present until the device is used to deliver therapeutic agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

Further details regarding a sterile tip that may be employed in embodiments of the delivery devices are provided in U.S. Provisional Patent Application Ser. No. 62/694,794; the disclosure of which is herein incorporated by reference.

The device may, where desired, include an antimicrobial element. The antimicrobial element may be any convenient element having antimicrobial properties and be positioned at one or more locations of the therapeutic agent delivery system. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the therapeutic agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol or other alcohols, and the like.

The active agent delivery system may, where desired, include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the tip in order to provide for at least aseptic conditions during contact of the device to the target tissue delivery site. The analgesic/anesthetic agent may be present in a holder, such as a matrix material, reservoir, etc. As with the therapeutic agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocaine, lidocaine, dubicaine, mepivacaine, bupivacaine, and the like; naturally-derived products, such as saxitoxin, neosaxitoxin, tetrodotoxin, menthol, eugenol, and cocaine, and the like; etc.

The active agent delivery devices of the invention may be configured for delivery of an active agent to a variety of target tissues and/or delivery sites therefore. Examples of target tissues include both external and internal sites, where internal delivery sites include those sites located in body cavities. External sites may include keratinized sites, as well as sites characterized by cutaneous membranes, mucous membranes, and tissue of the mucocutaneous zone. In some instances, the target tissue is one that accessed via an ocular tissue delivery site, where ocular tissue delivery sites of interest include a region that begins at the corneal limbus and extends anywhere from 1 mm to 10 mm posterior to the limbus, 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include cornea, conjunctiva, episclera, and sclera of the eye. Ocular tissue delivery sites of interest include those that provide for intravitreal injection therapy (IVT), retrobulbar injection therapy, subtenon injection therapy, subretinal injection therapy, suprachorodial injection, subconjunctival injection therapy, intracameral injection therapy, and the like.

As summarized above, the device includes a motorized actuator which is configured to move the plunger of the syringe during use. In these instances, the syringe and optional tissue contacting tip, which collectively may be referred to as the active agent delivery system, may be configured to be operably, and in some instances release-ably, engaged in a receiving space of an actuator component of the device. In these instances, the active agent delivery system may include a component of a locking element for release-ably engaging the active agent delivery system in a receiving space of the actuator component of the device. Any convenient locking mechanism may be employed, such as but not limited to: press fit, moveable latch, and the like. The locking element component of the locking element that is present on the active agent delivery component may vary, as desired, and is selected based on the companion element that is present on the actuator.

As such, embodiments of active agent delivery devices include an active agent delivery system and an actuator component, where the active agent delivery system is present in a receiving space, such as a syringe receiving space, of the actuator component and the actuator component includes an active agent delivery system actuator. In some instances, the active agent delivery system is release-ably engaged in the receiving space of the actuator component. Accordingly, in such instances the active agent delivery system is configured to be readily separable from the receiving space of the actuator component without in any way damaging the functionality of the actuator component, such that another active agent delivery system may be positioned in the receiving space of the actuator component. As such, the devices of the present invention are configured so that the actuator component can be sequentially employed with multiple different active agent delivery systems. Of interest are configurations in which the active agent delivery system can be manually operably positioned in the receiving space of the actuator component unit without the use of any tools. In some instances, the device further includes a locking element for release-ably engaging the active agent delivery system in the receiving space of the actuator component of the device. Any convenient locking mechanism may be employed, such as but not limited to: press fit, moveable latch, and the like.

Where a given device includes both an active agent delivery system and an actuator component, the active agent delivery system may further include one or more identifiers. When present, such identifiers may be present on one or more components of the active agent delivery system, such as the syringe, the tip, etc. In some instances, an identifier present on the active agent delivery system is an identifier that is configured to be read by an identifier reader of the actuator component of the device, e.g., as described below. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the therapeutic agent delivery system may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the active agent delivery system or active agent present therein, such as identity of the therapeutic agent (brand name and/or generic name), date of manufacture, date of expiry, source of manufacture, dosage amount, drug concentration, intended route of administration, handling and storage information, delivery volume, indication for use, lot number, etc.

As described above, the actuator component is configured to operably engage with an active agent delivery system such as described above, to produce an active agent delivery device of the invention. Aspects of actuator components according to embodiments of the invention include a body having a proximal end and a distal end, an active agent delivery system receiving space configured to be operably, and in some instances release-ably, engaged with an active agent delivery system, e.g., as described above. Further aspects of the actuator component include an active agent delivery system actuator configured to actuate an active agent delivery system. The actuator may further include one or more components of a pain mitigation system configured to mitigate pain in target tissue delivery site, and in some instances the actuator may include all of the components of a pain mitigation system.

The actuator is an element or subsystem that is configured to actuate the active agent delivery system so as to deliver an active agent to a target tissue delivery site. The nature of the active agent delivery system actuator may vary, e.g., depending on the nature of the active agent delivery system. For example, the active agent delivery system actuator may be configured to provide for control of one or more of angular position, linear position, velocity and acceleration of the needle. In some instances, the actuator, either alone or in conjunction with a guiding element of the active agent delivery system, is configured to provide for an angle of the needle relative the distal, tissue contacting end of the active agent delivery system, that ranges from 0 to 90, such as 75 to 90°. In some embodiments, the active agent delivery system is present in the device at a pre-determined angle, for example, 90 degrees to the biologic tissue when the cold tip is applied to the biologic tissue, so that when the device tip is placed on the eye abutting the limbus and causing very slight indentation of the ocular surface 360 degrees around the tip, the needle tip will reproducibly be inserted into the eye at a defined, safe angle posterior to the limbus of the eye to avoid the danger of striking the retina, zonules, or lens. In some instances, the actuator provides for a velocity of introduction of the injector into a target tissue delivery site that ranges from 0.1 to 100 mm/sec, such as 1 to 10 mm/sec, and including 3.5 to 9 mm/sec. The active agent delivery system actuator may be configured to provide for control of release of an active agent composition from the syringe. The actuator may be configured to provide for controlled removal of the needle from the target tissue delivery site. In some such instances, the actuator may be configured to withdraw the needle from a target tissue delivery site at a velocity ranging from 0.1 to 100 mm/sec, such as 1 to 10 mm/sec, and including 3.5 to 9 mm/sec. In some instances, the actuator is configured to prime the tissue injector, e.g., where the active agent delivery system includes an amount of gas, e.g., air (such as in the form of bubbles) and the actuator removes the gas from the system, e.g., by causing the gas to evacuate from the system via the needle. In some instances, the actuator is configured to sequentially move the drug container in a first priming motion and a second injection motion. In some instances, the actuator is further configured to withdraw the needle back into the device following injection of the active agent composition

The actuator may vary as desired. Examples of actuators that may be employed in embodiments of the invention and present in the actuator component include, but are not limited to: motorized actuators (including those that include a micro-motor). The motorized actuator may be configured to deliver a force ranging from 0.05N to 250N, such as 0.5N-50.0N. The motorized actuator may be configured to provide for rapid delivery of the viscous composition to the target site, such as the intravitreal site, where in some instances the rapid delivery time ranges from 0.1 to 60 seconds, such as 0.5 to 10 seconds. The functionality of the actuator may be controlled by one or more modules, as desired. The actuator can be configured to modify the speed and depth of drug injection.

In addition to the actuator, the actuator component may include one or more components of, including all of, a pain mitigation system. The actuator may provide for pain mitigation, such that the actuator is configured to alleviate pain associated with delivery of an active agent to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation). A pain mitigation system is a system that provides for pain alleviation during delivery of an active agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and, in some instances, is a system that provides for contact of a cold element (e.g., a cold tip or cold tissue engager (such as a tissue contacting tip, e.g., as described in greater detail below) with the target tissue delivery site. The cold element (which may be a component of a tissue engager, e.g., as described in greater detail below) of the cooling system may vary, and, in some instances, is an element that is configured to maintain a temperature of between −80° C. to +5° C., such as −20° C. to 0° C., such as −20° C. to −5° C. and including −15° C. to −5° C. when contacted with the target tissue delivery site. During a given delivery method, a tissue engager may maintain a constant temperature or cycle through one or more distinct temperature ranges, as desired. For example, a tissue engager may be configured to have a temperature that falls within a first range (e.g., as described above) to provide for desired cryoanesthesia during therapeutic agent delivery, and then cycle to a second, warmer temperature prior to removal of the device, such as a temperature ranging from 0 to −5° C., such as 0 to −2.5° C., including 0 to −1° C. Where the target tissue delivery site is an ocular tissue delivery site, e.g., as described elsewhere, delivery of cooling to cause rapid vasoconstriction enables a reduction in the occurrence of ocular surface bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems, low-temperature substance cooling systems, and the like.

In some instances, the pain mitigation anesthesia producing system is a thermoelectric cooling system, e.g., one that includes one or a combination of thermoelectric (Peltier) devices or units. While thermoelectric cooling systems employed in embodiments of devices of the invention may vary, in some instances the thermoelectric cooling systems include a cold tip that is configured to contact a target tissue delivery site (and therefore may also be referred to as a tissue engager), as well as one or more of a power source, a controller, a cooling power concentrator, one or more Peltier unit modules, and a heat sink (which may be a solid material or include one a fluid, such as a liquid, phase in a container. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Pat. No. 9,956,355; the disclosure of which is herein incorporated by reference.

In yet other instances, the cooling system may include a substance having a freezing temperature of 0° C. or lower. As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that deliver an anesthetic agent, such as but not limited to: sodium-channel blockers, e.g., as amino amides or amino esters (such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to, additional techniques such as electrical stimulation (Campbell and Taub, Arch Neurol. 1973; 28(5):347-350) vibration producing systems and the like.

As indicated above, in some instances, the actuator component may include a component of a locking element for release-ably engaging the active agent delivery system in the receiving space of the actuator component of the device. As reviewed above, any convenient locking mechanism may be employed, such as but not limited to: press fit or snap on, and the like. The locking element component of the locking element that is present on the actuator component may vary, as desired, and is selected based on the companion element that is present on the actuator.

In some instances, the actuator component includes an identifier reader for reading an identifier of an active agent delivery system release-ably engaged with the actuator component. The identifier reader may vary, as desired, depending on the nature of the identifier that is associated with the therapeutic agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID reader or NFC reader. The identifier reader, when present, is located on the actuator at a position such that it is reading relationship with the identifier of a therapeutic agent delivery component when release-ably engaged with the actuator.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the active agent delivery system is filled with the correct active agent, whether the active agent delivery component is expired, where the active agent delivery component is manufactured by an acceptable, authentic source; whether the active agent delivery component has been previously registered as lost, etc. In such instances, the reader may be coupled to an actuator control element that only enables one or more actuator components, such as the therapeutic agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a single to the controller that one or more of the actuator components should not be enabled.

Actuator components of the invention may further include a communications module, which module is operably coupled to one or more components of the actuator and provide for data transfer therefrom to another component, e.g., an external device, etc. The communications module may be configured to provide for the transfer of data in a wired or wireless mode, as desired. For example, the communications module may be configured to wirelessly transfer data, e.g., with a networked device, while be used, and then transfer data using a wired configuration when docked at a docking station, such as described below. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary, and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 100 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, a actuator component is updated to configure the unit to be used with a particular therapeutic agent delivery component. In this fashion, the same actuator component may be employed with two or more different therapeutic agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the therapeutic agent delivery system actuator will provide for actuation only if a switch is depressed continuously during the injection process. In some embodiments, there will be a safety mechanism to halt injection.

In some instances, the actuator components may include a display. By display is meant a visual display unit, which may include a screen that displays visual data in the form of images, lights, and/or text to a user. The screen may vary, where a screen type of interest is an LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, etc. The display may be configured to display a variety of different types of information to a user, where such information may include devices settings, including tip temperature, time of cooling application, therapeutic agent identification, and therapeutic agent expiration date, etc.

The entire active agent delivery device may be configured for single use, such that the entire active agent delivery device is disposable. Alternatively, one or more components of the active agent delivery device may be reusable. For example, the actuator component may be reusable while the active agent delivery system and components thereof, e.g., syringe, needle, tissue contacting tip, etc., may be single use.

The various device components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin copolymer (Crystal Zenith) and the like.

Active agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and, in some instances, may range from 0.05 to 3 pounds, such as 0.1 pounds to 1 pound. Handheld devices of the invention may have any convenient configuration, where examples of suitable handle configurations are further provided below.

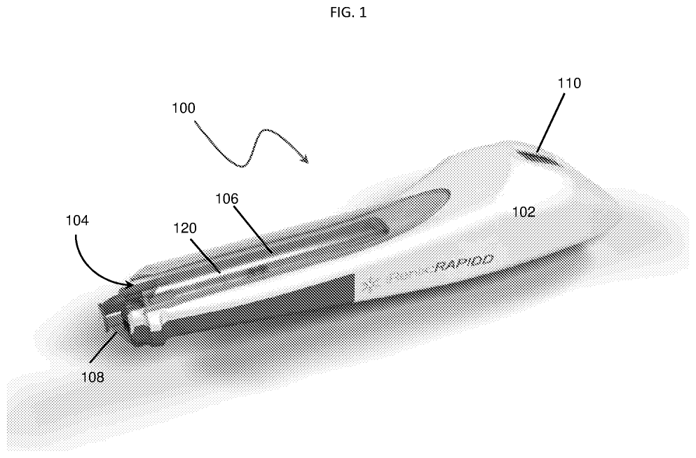

FIG. 1 provides a view of a delivery device according to any embodiment of the invention configured for delivery of a viscous active agent composition, e.g., as described above. As shown in FIG. 1, device 100 includes an actuator component 102 that includes an active agent delivery system receiving space 104 and a transparent cover for the receiving space 106. Also shown is the cooling tip 108 of a pain mitigation system of the actuator component 102. The actuator component 102 also includes an LED 110. Present in the receiving space 104 is active agent delivery system 120, which includes a tip 122 having a sterile, planar tissue contacting surface 124.

### Solid Formulation Delivery Devices

As summarized above, solid formulation active agent delivery devices are provided. As the devices are active agent delivery devices, they are configured to deliver an amount (e.g., dosage) of an active, e.g., therapeutic, agent to a target tissue, e.g., via a target delivery site, of a subject. As will be reviewed in greater detail below, the active agent is present in a solid formulation.

As summarized above, aspects of the devices include a sleeve that holds one or more solid formulations of the active agent. The sleeve includes a body, such as a tubular body, e.g., a calibrated cylindrical barrel (which may be fabricated from glass or plastic) having a first or proximal end and an orifice at a second or distal end, e.g., for providing passage of the active agent solid formulation from the inside of the sleeve into a needle which may be operably engaged to the distal end of the sleeve. The sleeve may include a seal on both ends to prevent passage of materials in or out of the sleeve until the time of activation. The seal in the distal end may be configured to be ruptured or removed prior to use. The seal may enable sterility of the solid drug formulation. The sleeve is configured to hold a desired number of solid formulations, including one or more, e.g., multiple solid formulations, e.g., 2 to 10 solid formulations. The sleeve may be fabricated from any convenient material, including glasses (e.g., type 1 class), plastics (such that the container is a polymeric container, e.g., cyclic olefin polymer (COP) and cyclic olefin copolymer (COC)), etc. Suitable materials include, but are not limited to, those described in published PCT application publication nos. WO 2013/178771; WO2015/173260; WO2017/087798 and WO2017/085253; the disclosures of which are herein incorporated by reference.

In some instances, the drug sleeve is configured to be terminally sterilized after drug is loaded therein. In some instances, the sleeve is not surface sterilized. As the sleeve is not surface sterilized, the outer surface of the sleeve is non-sterile. Non-sterile means any that must be considered to be not sterile or cannot be considered to be sterile, e.g., because of the history of the surface, such as the handling/processing history of the surface (for example as determined by standard medical procedures). In some instances, the non-sterile surface is one that has not been treated so that it is free of live bacteria or other microorganisms (for example, the surface has not undergone a sterilization procedure, such as a heat sterilization procedure (e.g., steam exposure) or chemical sterilization procedure, (e.g., exposure to ethylene oxide gas, exposure to hydrogen peroxide gas plasma, peracetic acid immersion, ozone exposure, etc.). In some instances, the non-sterilized surface has at least one live bacteria or other microorganism present thereon. While the outer surface of the sleeve is not sterile, the interior of the sleeve, as well as the contents thereof, e.g., a solid formulation of an agent, are sterile.

Any desired active agent solid formulation may be present in the sleeve, where an active agent solid formulation or composition may include a single active agent or combination of two or more different active agents, as desired. An active agent is any component that provides pharmacological activity or other direct effect in the diagnosis, cure, mitigation, treatment, or prevention of disease, or affects the structure or any function of the body of man or animals, and may be a liquid, gel, or solid. A type of active agent that may be present in a given active agent composition is a therapeutic agent, which is an agent that may be used in treating, remediating, or curing a disorder or disease. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; aminosterols such as squalamine; antihistamines such as H 1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; neuroprotective agents; hydrogels; glucose modulators, e.g., insulin; immune modulators, etc.

In addition to the active agent or agents, the solid formulations also include a matrix component, e.g., which may be configured to provide for controlled release of the active agent from the formulation over a desired time period. The matrix component may vary, where in some instances the matrix component is a polymeric matrix component, that includes one or more polymers, such as biodegradable polymers, e.g., a polymer selected from the group consisting of polylactides, poly(lactide-co-glycolides), polycaprolactones, and combinations thereof. Solid formulations may be a formulation such as described in U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; 6,699,493; 6,713,081 and 8,512,738; the disclosures of which are herein incorporated by reference.

Devices of the invention may also include a tip having a sterile tissue contacting surface. As the tissue contacting surface of the tip is sterile, as least the portion of the surface that contacts tissue during use of the device is free of live bacteria or other microorganisms. In some instances, the dimensions of the tissue contacting tip will be such that needle entry occurs at a predetermined distance from the corneal limbus (where distances may be as provided above) when the tissue contacting tip is placed on the ocular surface, where such configurations may negate any need for manual measurement for safe injection distance prior to procedure (intravitreal, intracameral etc.). As such, the tissue contacting tip may be configured to prevent contact with a tissue structure near a target tissue delivery site, e.g., an ocular lens or ocular retina where the target tissue delivery site is an ocular tissue delivery site. For example, the tissue contacting tip may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. While the tissue contacting surface may have any convenient configuration, in some instances the surface is planar. The shape of the tissue contacting surface may vary, where shapes of certain embodiments include circular, oval, triangular, rectangular, including square, hexagonal, octagonal, etc. The area of the tissue contacting surface may also vary, where in some instances the area ranges from 0.5 to 50 mm². In some instances, the tip can be operably shortened to allow for needle penetration, e.g., where the tip is a collapsible tip, e.g., that goes from being 15 mm to 10 mm beyond the distal end of the actuator, thus allowing the needle to be pushed outside the tip and penetrate biologic tissue. In some instances, the distance that the tip collapses may range from 2 to 10 mm, such as 2 to 5 mm.

Devices of the invention also include a needle operably coupled to the sleeve and the tip. The needle is configured to convey the active agent solid formulation from the sleeve to a target location, e.g., an intravitreal location, e.g., via a target tissue delivery site, such as topical ocular site. The needle may have any convenient dimensions, and, in some instances, has a gauge ranging from 20 to 35, such as 23 to 35, such as 27 to 35, e.g., 30 to 33, such as 29, 29½, 30, 31, 32, 33 and 34 gauge. The needle may be made of any convenient material, e.g., stainless steel, etc. At the proximal end of the needle may be a connector for operably connecting to the sleeve. Any convenient connector may be present, where the connector that may be present, such as a luer connector, e.g., a luer slip or luer lock connector.

In these embodiments where a tip is included, the needle is operably coupled to the sleeve and the tip. The sleeve or syringe may come pre-attached to the needle and tip, with a barrier in the sleeve preventing access of contaminants along the needle track and preventing drug loss from the sleeve. The proximal end of the needle may be operably engaged with the distal end of the sleeve such that the solid formulation inside of the sleeve passes through the distal opening of the sleeve and into the inside (i.e., bore) of the needle. The proximal end of the needle may be operably engaged with the distal end of the sleeve using any convenient configuration. Examples of suitable configurations include, but are not limited to, press fit configurations, luer fitting configurations, e.g., luer slip or luer lock, etc.

In addition to being operably coupled to the sleeve, the needle is also operably coupled to the tip that includes the sterile tissue contacting surface. In some instances, the tip is a structure having a proximal end attached to the needle, e.g., by a suitable fitting, and a distal end extending beyond the distal end of the needle, e.g., by a distance ranging from 2 to 20 mm, where the distal end includes the sterile tissue contacting surface. The tip may be made up of a single component or two or more components operably connected to each other. For example, a tip may include a first proximal component and a second distal component, where the distal component includes the sterile tissue contacting surface and the proximal component includes a fitting for operably coupling to the needle. The proximal component may further house the needle or a portion thereof, such that the proximal component may be considered to be a needle housing. The proximal and distal components may be configured to move relative to each other during use. The distance that the two components may move relative to each other may vary, where in some instances the distance ranges from 1 to 20 mm, such as 2 to 10 mm. In some instances, a locking mechanism maintains the distal end of the needle relative to the distal end of the tissue contacting tip prior to actuation. The locking mechanism may be releasable upon movement of the tissue contacting tip relative to the needle, such as a rotational movement, e.g., of the tissue contacting tip about the central longitudinal of the needle. For example, a tissue contacting tip may be turned, such as a quarter turn, relative to the associated needle which results in removing the lock and allowing the needle to be moved relative to the distal end of the tissue contacting tip. The distal, tissue contacting end of the tip may include an opening dimensioned to provide for passage of the distal end of the needle through the contacting tip during active agent delivery, thereby providing access of the needle directly to a target tissue. Upon actuation, the distal end of the needle moves relative to the distal end of the tissue contacting tip. The distance that the distal end of the needle extends beyond the distal end of the tissue contacting tip, and sterile surface thereof, during actuation and active agent delivery may vary, and, in some instances, ranges from 0.1 to 5 mm, such as 0.5 to 4.0 mm. While the dimensions of the opening, when present, may vary, in some instances the opening has a diameter sufficient to accommodate passage of a needle having a gauge, e.g., as described above. The distal end of the tissue contacting tip may in some instances be fabricated from a thermally conductive material, e.g., a metal or alloy thereof, such as in those instances where the distal end of the tissue contacting tip operatively engages the distal end of a cooling element of a cooling system of the actuator component, e.g., as described in greater detail below. Other suitable materials may also be employed, such as polymeric materials.

In some instances, the tissue contacting tip may include one or more filters. The filters may be configured to remove particles or other unwanted components present in the active agent composition prior to delivery to the target tissue delivery site. Such filters may be configured to inhibit passage of particles above a certain pore size from >0.1 μm to >50 μm, such as >5 μm. The one or more filters may be positioned at any convenient location in the therapeutic agent delivery system, e.g., at the exit from the container into the injector, at some point along the injector, at the distal end of the injector, etc.

Where desired, the distal, tissue contacting surface may include a removable cover, e.g., that is present until the device is used to deliver therapeutic agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

Further details regarding a sterile tip that may be employed in embodiments of the delivery devices are provided in U.S. Provisional Patent Application Ser. No. 62/694,794; the disclosure of which is herein incorporated by reference.

The device may, where desired, include an antimicrobial element. The antimicrobial element may be any convenient element having antimicrobial properties and be positioned at one or more locations of the therapeutic agent delivery system. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the therapeutic agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol or other alcohols, and the like.

The active agent delivery system may, where desired, may include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the tip in order to provide for at least aseptic conditions during contact of the device to the target tissue delivery site. The analgesic/anesthetic agent may be present in a holder, such as a matrix material, reservoir, etc. As with the therapeutic agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocalne, lidocaine, dubicaine, mepivacaine, bupivacaine, and the like; naturally-derived products, such as saxitoxin, neosaxitoxin, tetrodotoxin, menthol, eugenol, and cocaine, and the like; etc.

The active agent delivery devices of the invention may be configured for delivery of an active agent to a variety of target tissues and/or delivery sites therefore. Examples of target tissues include both external and internal sites, where internal delivery sites include those sites located in body cavities. External sites may include keratinized sites, as well as sites characterized by cutaneous membranes, mucous membranes, and tissue of the mucocutaneous zone. In some instances, the target tissue is one that accessed via an ocular tissue delivery site, where ocular tissue delivery sites of interest include a region that begins at the corneal limbus and extends anywhere from 1 mm to 10 mm posterior to the limbus, 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include cornea, conjunctiva, episclera, and sclera of the eye. Ocular tissue delivery sites of interest include those that provide for intravitreal injection therapy (IVT), retrobulbar injection therapy, subtenon injection therapy, subretinal injection therapy, suprachorodial injection, subconjunctival injection therapy, intracameral injection therapy, and the like.

In some instances, a given active agent delivery device includes only the sleeve and needle operably coupled to the tissue contacting tip, e.g., as described above. For example, the device may be configured for manual actuation, such that a user, such as a health care practitioner, can position the sterile tissue contacting surface of the tip onto a target location, e.g., a location of the surface of the eye, move the needle housing relative to the distal tip so as to move the needle through the eye surface into the eye, and then move a lever operably coupled to the sleeve so as to move a solid formulation of an active agent from the sleeve via the needle into the eye.

In yet other instances, the device may non-manual actuator which is configured to move operate the device during use. The sleeve may be configured to be operably, and in some instances release-ably, engaged in a receiving space of an actuator component of the device. In these instances, the sleeve may include a component of a locking element for release-ably engaging the active agent delivery system in a receiving space of the actuator component of the device. Any convenient locking mechanism may be employed, such as but not limited to: press fit, moveable latch, and the like. The locking element component of the locking element that is present on the active agent delivery component may vary, as desired, and is selected based on the companion element that is present on the actuator.

As such, embodiments of active agent delivery devices include an active agent delivery system (e.g., made up of a sleeve and needle, and optionally a tissue contacting tip) and an actuator component, where the active agent delivery system is present in a receiving space, such as a syringe receiving space, of the actuator component and the actuator component includes an active agent delivery system actuator. In some instances, the active agent delivery system is release-ably engaged in the receiving space of the actuator component. Accordingly, in such instances the active agent delivery system is configured to be readily separable from the receiving space of the actuator component without in any way damaging the functionality of the actuator component, such that another active agent delivery system may be positioned in the receiving space of the actuator component. As such, the devices of the present invention are configured so that the actuator component can be sequentially employed with multiple different active agent delivery systems. Of interest are configurations in which the active agent delivery system can be manually operably positioned in the receiving space of the actuator component unit without the use of any tools. In some instances, the device further includes a locking element for release-ably engaging the active agent delivery system in the receiving space of the actuator component of the device. Any convenient locking mechanism may be employed, such as but not limited to: press fit, moveable latch, and the like.

Where a given device includes both an active agent delivery system and an actuator component, the active agent delivery system may further include one or more identifiers. When present, such identifiers may be present on one or more components of the active agent delivery system, such as the syringe, the tip, etc. In some instances, an identifier present on the active agent delivery system is an identifier that is configured to be read by an identifier reader of the actuator component of the device, e.g., as described below. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the therapeutic agent delivery system may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the active agent delivery system or active agent present therein, such as identity of the therapeutic agent (brand name and/or generic name), date of manufacture, date of expiry, source of manufacture, dosage amount, drug concentration, intended route of administration, handling and storage information, delivery volume, indication for use, lot number, etc.

As described above, the actuator component is configured to operably engage with an active agent delivery system such as described above, to produce an active agent delivery device of the invention. Aspects of actuator components according to embodiments of the invention include a body having a proximal end and a distal end, an active agent delivery system receiving space configured to be operably, and in some instances release-ably, engaged with an active agent delivery system, e.g., as described above. Further aspects of the actuator component include an active agent delivery system actuator configured to actuate an active agent delivery system. The actuator may further include one or more components of a pain mitigation system configured to mitigate pain in target tissue delivery site, and in some instances the actuator may include all of the components of a pain mitigation system.

The actuator is an element or subsystem that is configured to actuate the active agent delivery system so as to deliver an active agent to a target tissue delivery site. The nature of the active agent delivery system actuator may vary, e.g., depending on the nature of the active agent delivery system. For example, the active agent delivery system actuator may be configured to provide for control of one or more of angular position, linear position, velocity and acceleration of the needle. In some instances, the actuator, either alone or in conjunction with a guiding element of the active agent delivery system, is configured to provide for an angle of the needle relative the distal, tissue contacting end of the active agent delivery system, that ranges from 0 to 90, such as 75 to 90°. In some embodiments, the active agent delivery system is present in the device at a pre-determined angle, for example, 90 degrees to the biologic tissue when the cold tip is applied to the biologic tissue, so that when the device tip is placed on the eye abutting the limbus and causing very slight indentation of the ocular surface 360 degrees around the tip, the needle tip will reproducibly be inserted into the eye at a defined, safe angle posterior to the limbus of the eye to avoid the danger of striking the retina, zonules, or lens. In some instances, the actuator provides for a velocity of introduction of the injector into a target tissue delivery site that ranges from 1 to 100 mm/sec, such as 1 to 10 mm/sec and including 3.5 to 9 mm/sec. The active agent delivery system actuator may be configured to provide for control of release of an active agent composition from the syringe. The actuator may be configured to provide for controlled removal of the needle from the target tissue delivery site. In some such instances, the actuator may be configured to withdraw the needle from a target tissue delivery site at a velocity ranging from 1 to 100 mm/sec, such as 1 to 10 mm/sec, and including 3.5 to 9 mm/sec. In some instances, the actuator is configured to prime the tissue injector, e.g., where the active agent delivery system includes an amount of gas, e.g., air (such as in the form of bubbles) and the actuator removes the gas from the system, e.g., by causing the gas to evacuate from the system via the needle. In some instances, the actuator is configured to prime the tissue injector by filling the dead space in a needle hub and needle shaft, without removing gas (i.e. air) from the syringe barrel. In some instances, the actuator is configured to sequentially move the drug container in a first priming motion and a second injection motion. In some instances, the actuator is further configured to withdraw the needle back into the device following injection of the active agent composition

The actuator may vary as desired. Examples of actuators that may be employed in embodiments of the invention and present in the actuator component include, but are not limited to: motorized actuators (including those that include a micro-motor), as well as non-motorized actuators, e.g., pneumatic powered actuators, hydraulically power actuators, spring-loaded actuators, manually operated actuators, e.g., plunger comprising actuators, and the like. The functionality of the actuator may be controlled by one or more modules, as desired. The actuator can be configured to modify the speed and depth of drug injection.

In addition to the actuator, the actuator component may include one or more components of, including all of, a pain mitigation system. The actuator may provide for pain mitigation, such that the actuator is configured to alleviate pain associated with delivery of an active agent to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation). A pain mitigation system is a system that provides for pain alleviation during delivery of an active agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and, in some instances, is a system that provides for contact of a cold element (e.g., a cold tip or cold tissue engager (such as a tissue contacting tip, e.g., as described in greater detail below) with the target tissue delivery site. The cold element (which may be a component of a tissue engager, e.g., as described in greater detail below) of the cooling system may vary, and, in some instances, is an element that is configured to maintain a temperature of between −80° C. to +5° C., such as −20° C. to 0° C. and including −20° C. to −5° C. and including −15° C. to −5° C. when contacted with the target tissue delivery site. During a given delivery method, a tissue engager may maintain a constant temperature or cycle through one or more distinct temperature ranges, as desired. For example, a tissue engager may be configured to have a temperature that falls within a first range (e.g., as described above) to provide for desired cryoanesthesia during therapeutic agent delivery, and then cycle to a second, warmer temperature prior to removal of the device, such as a temperature ranging from 0 to −5° C., such as 0 to −2.5° C., including 0 to −1° C. Where the target tissue delivery site is an ocular tissue delivery site, e.g., as described elsewhere, delivery of cooling to cause rapid vasoconstriction enables a reduction in the occurrence of ocular surface bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems, low-temperature substance cooling systems, and the like.

In some instances, the pain mitigation anesthesia producing system is a thermoelectric cooling system, e.g., one that includes one or a combination of thermoelectric (Peltier) devices or units. While thermoelectric cooling systems employed in embodiments of devices of the invention may vary, in some instances the thermoelectric cooling systems include a cold tip that is configured to contact a target tissue delivery site (and therefore may also be referred to as a tissue engager), as well as one or more of a power source, a controller, a cooling power concentrator, one or more Peltier unit modules, and a heat sink (which may be a solid material or include one a fluid, such as a liquid, phase in a container. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Pat. No. 9,956,355; the disclosure of which is herein incorporated by reference.

In yet other instances, the cooling system may include a substance having a freezing temperature of 0° C. or lower. As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that deliver an anesthetic agent, such as but not limited to: sodium-channel blockers, e.g., as amino amides or amino esters (such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to additional techniques such as electrical stimulation (Campbell and Taub, Arch Neurol. 1973; 28(5):347-350) vibration producing systems and the like.

As indicated above, in some instances, the actuator component may include a component of a locking element for release-ably engaging the active agent delivery system in the receiving space of the actuator component of the device. As reviewed above, any convenient locking mechanism may be employed, such as but not limited to: press fit or snap on, and the like. The locking element component of the locking element that is present on the actuator component may vary, as desired, and is selected based on the companion element that is present on the actuator.

In some instances, the actuator component includes an identifier reader for reading an identifier of an active agent delivery system release-ably engaged with the actuator component. The identifier reader may vary, as desired, depending on the nature of the identifier that is associated with the therapeutic agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID reader or NFC reader. The identifier reader, when present, is located on the actuator at a position such that it is reading relationship with the identifier of a therapeutic agent delivery component when release-ably engaged with the actuator.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the active agent delivery system is filled with the correct active agent, whether the active agent delivery component is expired, where the active agent delivery component is manufactured by an acceptable, authentic source; whether the active agent delivery component has been previously registered as lost, etc. In such instances, the reader may be coupled to an actuator control element that only enables one or more actuator components, such as the therapeutic agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a single to the controller that one or more of the actuator components should not be enabled.

Actuator components of the invention may further include a communications module, which module is operably coupled to one or more components of the actuator and provide for data transfer therefrom to another component, e.g., an external device, etc. The communications module may be configured to provide for the transfer of data in a wired or wireless mode, as desired. For example, the communications module may be configured to wirelessly transfer data, e.g., with a networked device, while be used, and then transfer data using a wired configuration when docked at a docking station, such as described below. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary, and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 100 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, an actuator component is updated to configure the unit to be used with a particular therapeutic agent delivery component. In this fashion, the same actuator component may be employed with two or more different therapeutic agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the therapeutic agent delivery system actuator will provide for actuation only if a switch is depressed continuously during the injection process. In some embodiments, there will be a safety mechanism to halt injection.

In some instances, the actuator components may include a display. By display is meant a visual display unit, which may include a screen that displays visual data in the form of images, lights, and/or text to a user. The screen may vary, where a screen type of interest is an LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, etc. The display may be configured to display a variety of different types of information to a user, where such information may include devices settings, including tip temperature, time of cooling application, therapeutic agent identification, and therapeutic agent expiration date, etc.

The entire active agent delivery device may be configured for single use, such that the entire active agent delivery device is disposable. Alternatively, one or more components of the active agent delivery device may be reusable. For example, the actuator component may be reusable while the active agent delivery system and components thereof, e.g., syringe, needle, tissue contacting tip, etc., may be single use.

The various device components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin copolymer (Crystal Zenith) and the like.

Active agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and, in some instances, may range from 0.05 to 3 pounds, such as 0.1 pounds to 1 pound. Handheld devices of the invention may have any convenient configuration.

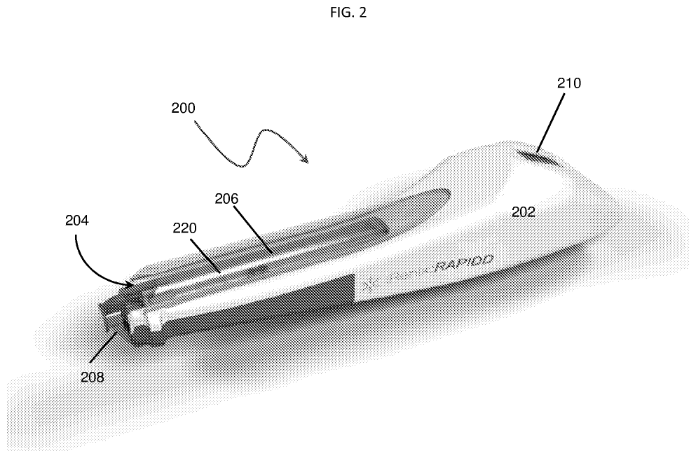

FIG. 2 provides a view of a hand-held therapeutic agent delivery device according to an embodiment of the invention. As shown in FIG. 2, device 200 includes an actuator component 202 that includes an active agent delivery system receiving space 204 and a transparent cover for the receiving space 206. Also shown is the cooling tip 208 of a pain mitigation system of the actuator component 202. The actuator component 202 also includes an LED 210. Present in the receiving space 204 is sleeve 220, which includes a solid active agent composition.

### Subcutaneous Active Agent Delivery Devices

As summarized above, subcutaneous delivery devices are provided. As the devices are subcutaneous delivery devices, they are configured to deliver an amount (e.g., dosage) of a subcutaneous composition, e.g., an aesthetic composition, to a subcutaneous location, e.g., via a topical dermal location, of a subject. By subcutaneous location is meant a location in the subcutis, i.e., the layer of skin directly below the dermis and epidermis.

As summarized above, the devices include a syringe containing a composition for subcutaneous delivery, a needle operably coupled to the syringe, a motorized actuator configured to deliver the composition from the syringe through the needle to a target subcutaneous site, and a pain mitigation system.

The term “syringe” is used in its conventional sense to refer to a device for injecting fluids. In some instances, a syringe includes a body, such as a tubular body, e.g., a calibrated cylindrical barrel (which may be fabricated from glass or plastic), having a plunger at a first or proximal end and an orifice at a second or distal end, e.g., for providing passage of the active agent composition from the inside of the syringe into a needle which may be operably engaged to the distal end of the container. The syringe is configured to hold a desired amount of an active agent composition. While the volume of the syringe may vary, in some instances the volume ranges from 0.1 to 5.0 ml, such as 0.1 to 1.50 ml, including 0.1 to 2.0 ml, e.g., 0.70 to 0.80 ml. In some instances, the volume of the syringe is sufficient to hold an amount of an active agent composition that is greater than the amount which is delivered to a target tissue delivery site during use of the device. While the magnitude of the excess may vary, in some instances the magnitude ranges from 110% to 500% of the delivered volume, such as 120% to 150%. Where the active agent composition is a non-gaseous composition, the amount of any gas, e.g., air, in the reservoir (and other components of the active agent delivery system) may be minimal, where in some instances the amount is 100 μl or less, such as 5 μl or less, 3 μl or less, 2 μl or less, or 1 μl or less. As indicated above, while the dosage of liquid active agent composition that is delivered to a target tissue by the devices may vary, in some instances the dosage ranges from 5 μl to 3500 μl, such as from 10 μl to 50 μl. In some instances, the syringe may include a dosing mark, e.g., an optically recognizable identifier that indicates delivery of a dosage of active agent composition. The dosage that is indicated by the dosing mark may vary, and, in some instances, ranges from 10 to 3500 μl, such as 1500 μl. In some instances, the drug may be delivered at custom amounts and the syringe may not have a dosing mark. The syringe may be fabricated from any convenient material, including glasses (e.g., type 1 class), plastics (such that the container is a polymeric container, e.g., cyclic olefin polymer (COP) and cyclic olefin copolymer (COC)), etc. Suitable materials include, but are not limited to, those described in published PCT application publication nos. WO 2013/178771; WO2015/173260; WO2017/087798 and WO2017/085253; the disclosures of which are herein incorporated by reference.

In some embodiments, the syringe is not surface sterilized. In some embodiments the syringe is surface sterilized post fill. In some instances, the drug is delivered in a vial and an off-the shelf syringe is used to draw up the medication into the syringe. In such instances, the syringe is not pre-filled, but prepared at the time of use or within a defined period prior to use, e.g., 24 hours or less before use. As the syringe is not surface sterilized, the outer surface of the syringe is non-sterile. Non-sterile means any that must be considered to be not sterile or cannot be considered to be sterile, e.g., because of the history of the surface, such as the handling/processing history of the surface (for example as determined by standard medical procedures). In some instances, the non-sterile surface is one that has not been treated so that it is free of live bacteria or other microorganisms (for example, the surface has not undergone a sterilization procedure, such as a heat sterilization procedure (e.g., steam exposure) or chemical sterilization procedure, (e.g., exposure to ethylene oxide gas, exposure to hydrogen peroxide gas plasma, peracetic acid immersion, ozone exposure, etc.). In some instances, the non-sterilized surface has at least one live bacteria or other microorganism present thereon. While the outer surface of the syringe is not sterile, the interior of the syringe, as well as the contents thereof, e.g., liquid active agent composition, are sterile.

Any desired subcutaneous composition may be present in a container (i.e., reservoir) of the syringe, where a subcutaneous composition may or may not include one or more active agents, as desired. An active agent is any component that provides pharmacological activity or other direct effect in the diagnosis, cure, mitigation, treatment, or prevention of disease, or affects the structure or any function of the body of man or animals, and may be a liquid, gel, or solid.

One type of subcutaneous composition of interest is an aesthetic composition. By aesthetic composition is meant a composition that is formulated to enhance the beauty of a subject to which it is administered, e.g., by reducing the appearance of wrinkles, enhancing skin tone, enhancing skin color, etc. Aesthetic compositions of interest include, but are not limited to, botulinum toxin compositions, dermal filler compositions, etc.

As summarized above, a given composition may include one or more active agents. A type of active agent that may be present in a given active agent composition is a therapeutic agent, which is an agent that may be used in treating, remediating, or curing a disorder or disease. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids, such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; aminosterols such as squalamine; antihistamines such as H 1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; glucose modulators, e.g., insulin; immune modulators; etc.

The active agent or agents are present in the active agent composition in a therapeutically effective amount. The amount of a given active in an active agent composition may vary, e.g., depending on the particular active agent, target condition, etc., where in some instances the amount of a given active agent ranges from 0.0005 to 10000 mg/ml, such as 10 to 500 mg/ml and including 25 to 200 mg/ml.

In addition to the active agent or agents, a given active agent composition include pharmaceutically acceptable delivery vehicle, e.g., a pharmaceutically acceptable aqueous vehicle. For pharmaceutically acceptable aqueous vehicles, in addition to water, the aqueous delivery vehicle may include a number of different components, including but not limited to: salts, buffers, preservatives, solubility enhancers, viscosity modulators, colorants, etc. Suitable aqueous vehicles include sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, viscosity modifiers, emulsifiers and stabilizers, antimicrobial agents, pH adjusting agents. The viscosity of a given active agent composition may vary. In some instances, the viscosity ranges from 0.5 to 5000 centipoise, such as 50 to 500 centipoise.

While the dosage of subcutaneous composition that is delivered to a target subcutaneous location by the devices may vary, in some instances the dosage ranges from 5 μl to 7500 μl, e.g., 5 μl to 3500 μl such as from 10 μl to 1500 μl and including 10 μl to 500 μl.

A given syringe may include in the container or reservoir thereof a single subcutaneous composition or two or more distinct subcutaneous compositions. The two or more subcutaneous compositions may differ from each other in terms of active agent(s), delivery vehicle, viscosity, etc. Where a given syringe includes two or more distinct subcutaneous compositions, the distinct subcutaneous composition may be separated from each other by a barrier, such as a frangible barrier, such that each distinct active agent composition is present in its own chamber of the syringe. In such instances, the syringe may have two or more chambers, e.g., one for each distinct active agent composition container therein. During delivery, the barrier(s) may be disrupted to provide for combination of the active agent compositions. For example, where two chambers are separated from each other by a frangible barrier, movement of the plunger during active agent composition delivery may disrupt the frangible barrier, allowing for mixing of the two or more active agent compositions. Where desired, a barrier disrupting mechanism may be employed, e.g., a barrier piercing mechanism, etc.

A given device may include a single syringe filed with one or more active agent compositions, e.g., as described above, or two or more syringes each filled with one or more active agent compositions, such as a device that includes two syringes each filled with a different active agent composition. Where a device includes two or more syringes each filled with a different active agent composition, the differing active agent compositions may differ from each other in terms of active agent(s), delivery vehicle, viscosity, etc. In these embodiments, the devices may be configured to deliver the active agent compositions contained in the distinct syringes as separate compositions to the target tissue, or they may be configured to combine the active agent compositions of the disparate syringes prior to delivery to a target tissue. In embodiments where the devices are configured to deliver the active agent compositions contained in the distinct syringes as separate compositions to the target tissue, each syringe may be operatively coupled to its own needle during active agent composition delivery. Alternatively, each syringe may be operatively coupled to a single needle having a distinct internal lumen or passageway for the contents of each syringe, such as needle having a central divider that defines two passageways fluidically isolated from each other and operably coupled to a syringe of a dual syringe system. In embodiments where the devices are configured to combine the active agent compositions of the disparate syringes prior to delivery to a target tissue, the syringes may be operably coupled to single needle having a single passageway during delivery, such that the contents of the distinct syringes mix upon passage through the needle. Where desired, a mixing element may be provided in the device, e.g., at the interface of the syringes and the needle.

Where desired, devices of the invention may also include a tip having a sterile tissue contacting surface. In such embodiments, as the tissue contacting surface of the tip is sterile, as least the portion of the surface that contacts tissue during use of the device is free of live bacteria or other microorganisms. In some instances, the dimensions of the tissue contacting tip will be such that needle entry occurs at a predetermined distance from a biological structure such as the eyebrow or nasal bridge. As such, the tissue contacting tip may be configured to prevent contact with a tissue structure near a target tissue delivery site, e.g., injection into the eye or orbit. For example, the tissue contacting tip may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. While the tissue contacting surface may have any convenient configuration, in some instances the surface is planar. The shape of the tissue contacting surface may vary, where shapes of certain embodiments include circular, oval, triangular, rectangular, including square, hexagonal, octagonal, etc. The area of the tissue contacting surface may also vary, where in some instances the area ranges from 0.5 to 50 mm², e.g., 0.25 to 1.0 mm², such as 0.5 mm². In some instances, a small tip surface diameter allows for better visualization of the cooling tip and needle, facilitating ease of use for subcutaneous injections on the face. In some embodiments, the tip is convex in shape, enabling cooling contact with tissue even when the device is used at an angle to facilitate drug delivery on curved surfaces or shallower needle penetration. In some instances, the tip will be collapsible, so that an actuator operably shortens the tip, allowing for an integrated needle to be exposed outside the tip.

Devices of the invention also include a needle operably coupled to the syringe and, optionally, the tip when present. The needle is configured to convey the active agent composition from the syringe to a target tissue, e.g., via a target tissue delivery site. The needle may have any convenient dimensions, and, in some instances, has a gauge ranging from 20 to 41, such as 23 to 35, such as 27 to 35, e.g., 30 to 33, such as 29, 29½, 30, 31, 32, 33 and 34 gauge. The needle may include a single lumen or two or more separate lumens container within a common core, as desired, e.g., where the needle includes two separate lumens contained within a common core and has a gauge ranging from 23 to 30. The needle may be made of any convenient material, e.g., stainless steel, etc. At the proximal end of the needle may be a connector for operably connecting to the syringe. Any convenient connector may be present, where connector may be present, such as a luer connector, e.g., a luer slip or luer lock connector.

As summarized above, the needle is operably coupled to the syringe and, optionally, the tip. The proximal end of the needle may be operably engaged with the distal end of the syringe such that liquid composition inside of the syringe passes through the distal opening of the syringe and into the inside (i.e., bore) of the needle. The proximal end of the needle may be operably engaged with the distal end of the syringe using any convenient configuration. Examples of suitable configurations include, but are not limited to, press fit configurations, luer fitting configurations, e.g., luer slip or luer lock, etc.

In addition to being operably coupled to the syringe, when present the needle is also operably coupled to the tip that includes the sterile tissue contacting surface. In some instances, the tip is a structure having a proximal end attached to the needle, e.g., by a suitable fitting, and a distal end extending beyond the distal end of the needle, e.g., by a distance ranging from 2 to 20 mm, where the distal end includes the sterile tissue contacting surface. The tip may be made up of a single component or two or more components operably connected to each other. For example, a tip may include a first proximal component and a second distal component, where the distal component includes the sterile tissue contacting surface and the proximal component includes a fitting for operably coupling to the needle. The proximal component may further house the needle or a portion thereof, such that the proximal component may be considered to be a needle housing. The proximal and distal components may be configured to move relative to each other during use. The distance that the two components may move relative to each other may vary, where in some instances the distance ranges from 1 to 20 mm, such as 2 to 10 mm. In some instances, a locking mechanism maintains the distal end of the needle relative to the distal end of the tissue contacting tip prior to actuation. The locking mechanism may be releasable upon movement of the tissue contacting tip relative to the needle, such as a rotational movement, e.g., of the tissue contacting tip about the central longitudinal of the needle. For example, a tissue contacting tip may be turned, such as a quarter turn, relative to the associated needle which results in removing the lock and allowing the needle to be moved relative to the distal end of the tissue contacting tip. The distal, tissue contacting end of the tip may include an opening dimensioned to provide for passage of the distal end of the needle through the contacting tip during active agent delivery, thereby providing access of the needle directly to a target tissue. Upon actuation, the distal end of the needle moves relative to the distal end of the tissue contacting tip. The distance that the distal end of the needle extends beyond the distal end of the tissue contacting tip, and sterile surface thereof, during actuation and active agent delivery may vary, and, in some instances, ranges from 0.1 to 5 mm, such as 0.5 to 4.0 mm. While the dimensions of the opening, when present, may vary, in some instances the opening has a diameter sufficient to accommodate passage of a needle having a gauge, e.g., as described above. The distal end of the tissue contacting tip may in some instances be fabricated from a thermally conductive material, e.g., a metal or alloy thereof, such as in those instances where the distal end of the tissue contacting tip operatively engages the distal end of a cooling element of a cooling system of the actuator component, e.g., as described in greater detail below. Other suitable materials may also be employed, such as polymeric materials. A given tip may be configured to be operably coupled to a single needle, or two or more needles. For example, a given tip may be configured to operably couple to two needles, e.g., where a device includes two syringes, such as described above.

In some instances, the tissue contacting tip may include one or more filters. The filters may be configured to remove particles or other unwanted components present in the active agent composition prior to delivery to the target tissue delivery site. Such filters may be configured to inhibit passage of particles above a certain pore size from >0.1 μm to >50 μm, such as >5 μm. The one or more filters may be positioned at any convenient location in the therapeutic agent delivery system, e.g., at the exit from the container into the injector, at some point along the injector, at the distal end of the injector, etc.

Where desired, the distal, tissue contacting surface may include a removable cover, e.g., that is present until the device is used to deliver therapeutic agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

Further details regarding a sterile tip that may be employed in embodiments of the delivery devices are provided in U.S. Provisional Patent Application Ser. No. 62/694,794; the disclosure of which is herein incorporated by reference.

The device may, where desired, include an antimicrobial element. The antimicrobial element may be any convenient element having antimicrobial properties and be positioned at one or more locations of the therapeutic agent delivery system. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the therapeutic agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol, Purite, or other alcohols, and the like.

The active agent delivery system may, where desired, include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the tip in order to provide for at least aseptic conditions during contact of the device to the target tissue delivery site. The analgesic/anesthetic agent may be present in a holder, such as a matrix material, reservoir, etc. As with the therapeutic agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocalne, lidocaine, dubicaine, mepivacaine, bupivacaine, and the like; naturally-derived products, such as saxitoxin, neosaxitoxin, tetrodotoxin, menthol, eugenol, and cocaine, and the like; device tip vibration, etc.

The delivery devices of the invention may be configured for delivery of a subcutaneous composition to a variety of target tissues and/or delivery sites therefore. Examples of target tissues include both external and internal sites, where internal delivery sites include those sites located in body cavities. External sites may include keratinized sites, as well as sites characterized by cutaneous membranes, mucous membranes, and tissue of the mucocutaneous zone. In some instances, the target tissue is a subcutaneous location that accessed via a topical delivery site, maxillofacial location.

As summarized above, the device includes a motorized actuator which is configured to move the plunger of the syringe during use. In these instances, the syringe and optional tissue contacting tip, which collectively may be referred to as the active agent delivery system, may be configured to be operably, and in some instances release-ably, engaged in a receiving space of an actuator component of the device. In these instances, the active agent delivery system may include a component of a locking element for release-ably engaging the active agent delivery system in a receiving space of the actuator component of the device. Any convenient locking mechanism may be employed, such as but not limited to: press fit, moveable latch, and the like. The locking element component of the locking element that is present on the active agent delivery component may vary, as desired, and is selected based on the companion element that is present on the actuator.

As such, embodiments of active agent delivery devices include an active agent delivery system and an actuator component, where the active agent delivery system is present in a receiving space, such as a syringe receiving space, of the actuator component and the actuator component includes an active agent delivery system actuator. In some instances, the active agent delivery system is release-ably engaged in the receiving space of the actuator component. Accordingly, in such instances the active agent delivery system is configured to be readily separable from the receiving space of the actuator component without in any way damaging the functionality of the actuator component, such that another active agent delivery system may be positioned in the receiving space of the actuator component. As such, the devices of the present invention are configured so that the actuator component can be sequentially employed with multiple different active agent delivery systems. Of interest are configurations in which the active agent delivery system can be manually operably positioned in the receiving space of the actuator component unit without the use of any tools. In some instances, the device further includes a locking element for release-ably engaging the active agent delivery system in the receiving space of the actuator component of the device. Any convenient locking mechanism may be employed, such as but not limited to: press fit, moveable latch, and the like.

Where a given device includes both an active agent delivery system and an actuator component, the active agent delivery system may further include one or more identifiers. When present, such identifiers may be present on one or more components of the active agent delivery system, such as the syringe, the tip, etc. In some instances, an identifier present on the active agent delivery system is an identifier that is configured to be read by an identifier reader of the actuator component of the device, e.g., as described below. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the therapeutic agent delivery system may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the active agent delivery system or active agent present therein, such as identity of the therapeutic agent (brand name and/or generic name), date of manufacture, date of expiry, source of manufacture, dosage amount, drug concentration, intended route of administration, handling and storage information, delivery volume, indication for use, lot number, etc.

As described above, the actuator component is configured to operably engage with an active agent delivery system such as described above, to produce an active agent delivery device of the invention. Aspects of actuator components according to embodiments of the invention include a body having a proximal end and a distal end, an active agent delivery system receiving space configured to be operably, and in some instances release-ably, engaged with an active agent delivery system, e.g., as described above. Further aspects of the actuator component include an active agent delivery system actuator configured to actuate an active agent delivery system. The actuator may further include one or more components of a pain mitigation system configured to mitigate pain in target tissue delivery site, and in some instances the actuator may include all of the components of a pain mitigation system.

The actuator is an element or subsystem that is configured to actuate the active agent delivery system so as to deliver an active agent to a target tissue delivery site. The nature of the active agent delivery system actuator may vary, e.g., depending on the nature of the active agent delivery system. For example, the active agent delivery system actuator may be configured to provide for control of one or more of angular position, linear position, velocity and acceleration of the needle. In some instances, the actuator, either alone or in conjunction with a guiding element of the active agent delivery system, is configured to provide for an angle of the needle relative the distal, tissue contacting end of the active agent delivery system, that ranges from 0 to 90, such as 75 to 90°. In some embodiments, the active agent delivery system is present in the device at a pre-determined angle, for example, 90 degrees to the biologic tissue when the cold tip is applied to the biologic tissue, so that when the device tip is placed on the tissue and causing very slight indentation of the tissue 360 degrees around the tip, the needle tip will reproducibly be inserted into the tissue at a defined, safe angle. In some instances, the actuator provides for a velocity of introduction of the injector into a target tissue delivery site that ranges from 0.1 to 100 mm/sec, such as 1 to 10 mm/sec, and including 3.5 to 9 mm/sec. The active agent delivery system actuator may be configured to provide for control of release of an active agent composition from the syringe. The actuator may be configured to provide for controlled removal of the needle from the target tissue delivery site. In some such instances, the actuator may be configured to withdraw the needle from a target tissue delivery site at a velocity ranging from 0.1 to 100 mm/sec, such as 1 to 10 mm/sec, and including 3.5 to 9 mm/sec. In some instances, the actuator is configured to prime the tissue injector, e.g., where the active agent delivery system includes an amount of gas, e.g., air (such as in the form of bubbles) and the actuator removes the gas from the system, e.g., by causing the gas to evacuate from the system via the needle. In some instances, the actuator is configured to sequentially move the drug container in a first priming motion and a second injection motion. In some instances, the actuator is configured to repeatedly move the same distance, delivering more than one injection of the same volume. In some instances, the device can deliver a number of doses, such as 2 to 40, and including 2 to 25 doses. In some instances, the device can be programed during use to deliver a variety of different doses, such as 0.01 ml to 10 ml, including 0.05 ml to 0.5 ml. In some instances, the actuator is further configured to withdraw the needle back into the device following injection of the active agent composition

The actuator may vary as desired. Examples of actuators that may be employed in embodiments of the invention and present in the actuator component include, but are not limited to: motorized actuators (including those that include a micro-motor). The motorized actuator may be configured to deliver a force ranging from 0.05N to 250N, such as 0.5N-50.0N. The motorized actuator may be configured to provide for rapid delivery of the viscous composition to the target site, such as the intravitreal site, where in some instances the rapid delivery time ranges from 0.1 to 60 seconds, such as 0.5 to 10 seconds. The functionality of the actuator may be controlled by one or more modules, as desired. The actuator can be configured to modify the speed and depth of drug injection.

In addition to the actuator, the actuator component may include one or more components of, including all of, a pain mitigation system. The actuator may provide for pain mitigation, such that the actuator is configured to alleviate pain associated with delivery of an active agent to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation). A pain mitigation system is a system that provides for pain alleviation during delivery of an active agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and, in some instances, is a system that provides for contact of a cold element (e.g., a cold tip or cold tissue engager (such as a tissue contacting tip, e.g., as described in greater detail below) with the target tissue delivery site. The cold element (which may be a component of a tissue engager, e.g., as described in greater detail below) of the cooling system may vary, and, in some instances, is an element that is configured to maintain a temperature of between −80° C. to +5° C., such as −20° C. to 0° C., such as −20° C. to −5° C. and including −15° C. to −5° C. when contacted with the target tissue delivery site. During a given delivery method, a tissue engager may maintain a constant temperature or allow gradual warming, or cycle through one or more distinct temperature ranges, as desired. For example, a tissue engager may be configured to have a temperature that falls within a first range (e.g., as described above) to provide for desired cryoanesthesia during therapeutic agent delivery, and then cycle to a second, warmer temperature prior to removal of the device, such as a temperature ranging from 0 to −5° C., such as 0 to −2.5° C., including 0 to −1° C. Where the target tissue delivery site is a cutaneous site, delivery of cooling to cause rapid vasoconstriction enables a reduction in the occurrence of tissue bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems, low-temperature substance cooling systems, and the like.

In some instances, the pain mitigation anesthesia producing system is a thermoelectric cooling system, e.g., one that includes one or a combination of thermoelectric (Peltier) devices or units. While thermoelectric cooling systems employed in embodiments of devices of the invention may vary, in some instances the thermoelectric cooling systems include a cold tip that is configured to contact a target tissue delivery site (and therefore may also be referred to as a tissue engager), as well as one or more of a power source, a controller, a cooling power concentrator, one or more Peltier unit modules, and a heat sink (which may be a solid material or include one a fluid, such as a liquid, phase in a container. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Pat. No. 9,956,355; the disclosure of which is herein incorporated by reference.

In yet other instances, the cooling system may include a substance having a freezing temperature of 0° C. or lower. As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that delivers an anesthetic agent, such as but not limited to: sodium-channel blockers, e.g., as amino amides or animo esters (such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to, additional techniques such as electrical stimulation (Campbell and Taub, Arch Neurol. 1973; 28(5):347-350), vibration producing systems, and the like.

As indicated above, in some instances, the actuator component may include a component of a locking element for release-ably engaging the active agent delivery system in the receiving space of the actuator component of the device. As reviewed above, any convenient locking mechanism may be employed, such as but not limited to: press fit or snap on, and the like. The locking element component of the locking element that is present on the actuator component may vary, as desired, and is selected based on the companion element that is present on the actuator.

In some instances, the actuator component includes an identifier reader for reading an identifier of an active agent delivery system release-ably engaged with the actuator component. The identifier reader may vary, as desired, depending on the nature of the identifier that is associated with the therapeutic agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID or NFC reader. The identifier reader, when present, is located on the actuator at a position such that it is reading relationship with the identifier of a therapeutic agent delivery component when release-ably engaged with the actuator.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the active agent delivery system is filled with the correct active agent, whether the active agent delivery component is expired, where the active agent delivery component is manufactured by an acceptable, authentic source; whether the active agent delivery component has been previously registered as lost, etc. In such instances, the reader may be coupled to an actuator control element that only enables one or more actuator components, such as the therapeutic agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a single to the controller that one or more of the actuator components should not be enabled.

Actuator components of the invention may further include a communications module, which module is operably coupled to one or more components of the actuator and provide for data transfer therefrom to another component, e.g., an external device, etc. The communications module may be configured to provide for the transfer of data in a wired or wireless mode, as desired. For example, the communications module may be configured to wirelessly transfer data, e.g., with a networked device, while be used, and then transfer data using a wired configuration when docked at a docking station, such as described below. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 1000 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, a actuator component is updated to configure the unit to be used with a particular therapeutic agent delivery component. In this fashion, the same actuator component may be employed with two or more different therapeutic agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the therapeutic agent delivery system actuator will provide for actuation only if a switch is depressed continuously during the injection process. In some embodiments, there will be a safety mechanism to halt injection.

In some instances, the actuator components may include a display. By display is meant a visual display unit, which may include a screen that displays visual data in the form of images, lights, and/or text to a user. The screen may vary, where a screen type of interest is an LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, etc. The display may be configured to display a variety of different types of information to a user, where such information may include devices settings, including tip temperature, time of cooling application, therapeutic agent identification, and therapeutic agent expiration date, volume of drug to be delivered etc. In an embodiment, the device screen is interactive with touch sensing technology, and enables the user to select different injection speeds, angles of injection, dose volumes and the like. The device includes a controller to operably manage the various injection functions as described above. The controller helps to maintain precise control of anesthesia activities in addition to drug delivery activities of the device. In some embodiments the device will have activation buttons that enable the modification of various factors (including, but not limited to, depth of injection, angle of penetration, speed of injection, and injection volume).

The entire active agent delivery device may be configured for single use, such that the entire active agent delivery device is disposable. Alternatively, one or more components of the active agent delivery device may be reusable. For example, the actuator component may be reusable while the active agent delivery system and components thereof, e.g., syringe, needle, tissue contacting tip, etc., may be single use.

The various device components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin copolymer (Crystal Zenith) and the like.

Active agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and, in some instances, may range from 0.01 to 3 pounds, such as 0.05 pounds to 1 pound, including 0.1 to 1 pound. Handheld devices of the invention may have any convenient configuration, where examples of suitable handle configurations are further provided below.

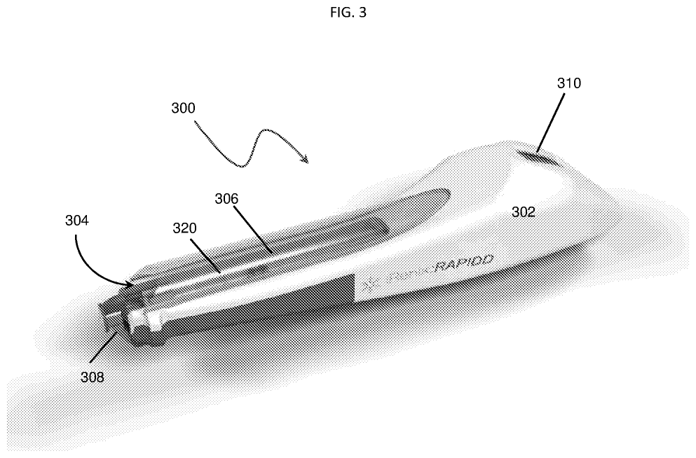

FIG. 3 provides a view of a hand-held therapeutic agent delivery device according to an embodiment of the invention. As shown in FIG. 3, device 300 includes an actuator component 302 that includes an active agent delivery system receiving space 304 and a transparent cover for the receiving space 306. Also shown is the cooling tip 308 of a pain mitigation system of the actuator component 302. The actuator component 302 also includes an LED 310. Present in the receiving space 304 is active agent delivery system 320, which includes a tip 322 having a sterile, planar tissue contacting surface 324.

### Docking Stations and Systems Including the Same

Aspects of the invention include docking stations that are configured to dock an actuator component, and systems that include a docking station and an actuator component. A docking station is a base unit or analogous device that is configured to engage with an actuator component, e.g., as described above. When engaged with an actuator component, the docking station may perform one or more functionalities, which functionalities may include, but are not limited to: maintaining a pain mitigation system in a desirable state (for example maintain a cooling system at a desired temperature); transferring data between the actuator component and an external device; sanitizing the distal end of an actuator component; recharging a power source of an actuator component; communicating with a computer, server, or database, and the like. The docking station may include a single actuator component dock, (i.e., a site or location configured to engagingly receive an actuator), or two or more actuator component docks, such that the number of actuator docks in a docking station may, in some instances, range from 1 to 6, such as 2 to 4. Docking stations of the invention may have any convenient configuration. Docking stations may be configured as table top devices, wall mounted devices, floor devices, etc., as desired. The component docks may be configured to only hold a cooling-power concentrator, which part can then be operably coupled to the device when another cooling power-concentrator is no longer at the desired temperature. The docking station may have several ports to charge several devices simultaneously, and may be configured to cool different devices that utilize the same mechanism of cooling or different mechanisms of cooling. In an embodiment, the base station may cool stand-alone cooling units that can be clipped into the device.

In order to provide different desired functionalities, the docking station may include a number of a different subsystems or components. For example, a docking station may include a cooling system, e.g., that is configured to maintain the temperature of docked actuator and/or therapeutic agent delivery components in a desired range. Examples of suitable cooling systems include, but are not limited, those described above. The docking station may include a communications module, e.g., for mediating data transfer between docked actuator and/or therapeutic agent delivery components and a module of the docking stations and/or an external device. The docking station may include a power module, e.g., for recharging a power source of a docked actuator. The docking station may include detector, e.g., for detecting docked actuator and/or therapeutic agent delivery components. The docking station may include an identifier reader, e.g., for reading an identifier on a docked actuator and/or therapeutic agent delivery components, such as an identifier reader as described above. Where desired, the docking station may include an identifier, such as described above.

In addition to docking stations, e.g., as described above, aspects of the invention further include docking systems. Docking systems include a docking station having one or more actuator components docked therewith.

### Smart Device Configurations

In some instances, the active agent delivery system includes an identifier and the actuator component includes an identifier reader, e.g., as described above, such that the device may be viewed as a “smart” device. In such embodiments, a variety of different types of information may be stored on the identifier. Reading of the identifier by the identifier reader, e.g., when the therapeutic agent delivery component is release-ably engaged to the actuator component, transfers the information to the actuator component.

In some instances, the identifier includes active agent delivery component information. Active agent delivery component information is information or data about the active agent delivery component itself. Such information may include active agent delivery component historical information. Historical information is information about the nature of the active agent delivery component and/or one or more past events experienced by the active agent delivery component. Historical information includes, but is not limited to: an active agent identifier (e.g., the name of the therapeutic agent (or a proxy thereof) contained in the active agent delivery component), manufacturing lot number for active agent and/or active agent delivery component, active agent delivery component handling information (e.g., information about the supply channel through which the active agent delivery component has passed), active agent delivery component dose, concentration, and/or volume, and active agent delivery component expiration date, chain of custody information (e.g., shipment tracking information including time and geographical information, and temperature information over time, such as any storage temperature excursions that may have occurred, etc.) and the like. As such, historical information may include information about a particular active agent contained in an active agent delivery component. Such information may include, but is not limited to, identity of the active agent (brand name and/or generic name), date of manufacture, date of expiry, source of manufacture, dosage amount, drug concentration, intended route of administration, handling and storage information, delivery volume, indication for use, lot number, etc.

In some instances, the identifier includes active agent delivery component information that is active agent delivery component use information. Active agent delivery component use information is information or data about the actual use of the active agent delivery component, e.g., the actual employment of that active agent delivery component to deliver an active agent to a target delivery site. Such information may vary, and may include use date information (i.e., information about the data, time, etc., at which the component was used); administration information, e.g., confirmation that actual delivery to a subject occurred); identity of the subject to which the therapeutic agent was administer; condition of the subject for which the therapeutic agent was administered, etc.

As will be appreciated by the skilled artisan, this information could be stored directly on the identifier, or could be looked up in a linked database using the identifier information.

Smart device embodiments, e.g., as described above, allow for one or more desirable capabilities, including but not limited to inventory management capabilities, enhanced therapeutic capabilities, medical record history capabilities, data analytics capabilities, and the like. For example, a variety of different inventory management capabilities are provided by smart device embodiments, including automated reordering of therapeutic agent delivery component by a user (e.g., according to user preset preferences), tracking of individual therapeutic agent delivery components (e.g., to manage lost, stolen, or expired goods components), and the like. A variety of different enhanced therapeutic capabilities are provided by smart device embodiments, including auto-generation of procedure notes, communication with existing electronic medical records for integration in a patient chart, sending data on drug/dose/route for documentation purposes and/or billing purposes, aggregating data on drug/dose/route for market research and analytics, facilitating documentation for reimbursement, and the like. Examples of capabilities provided by smart device embodiments, e.g., as described herein, are further provided in Published United States Patent Application Publication Nos. 20160030683; 20170098058; 20170119969; 20170124284 and 20170124285; the disclosures of which capabilities are incorporated herein by references.

Examples of capabilities provided by smart device embodiments, e.g., as described herein, are further provided in PCT Application Serial No. PCT/US2018/037157; the disclosure of which is herein incorporated by reference.

### Methods

Aspects of the invention further include methods of delivering an active agent to a target tissue delivery site of a subject, e.g., by using an active agent delivery device of the invention. Aspects of the methods may include: contacting a distal end of an active agent delivery device, e.g., as described above, to the target tissue delivery site; and actuating the active agent delivery system to deliver a dosage of an active agent composition to a target site.

As reviewed above, the target tissue delivery site may vary. Examples of target tissue delivery sites include both external and internal delivery sites, wherein internal delivery sites include those sites located in body cavities. External sites include keratinized sites, as well as sites characterized by cutaneous membranes, mucous membranes, and tissue of the mucocutaneous zone. In some instances, the target tissue delivery site is an ocular site, where ocular sites of interest include a region that begins at the corneal limbus and extends anywhere from 1 mm to 10 mm posterior to the limbus, such as 2 mm to over 8 mm posterior to the limbus. In some instances, the area of interest includes the cornea and the corneal limbus.

To contact the distal end of the device with the target tissue site, the device may be manipulated so that the distal end of the device contacts the target tissue site. Where desired, contact of the distal end with the target tissue delivery site may be maintained by urging the distal end against the target tissue delivery site with moderate force.

As described above, embodiments of devices may include a pain mitigation system. When present, the pain mitigation system may be actuated to mitigate pain at the target tissue delivery site. Depending on the nature of the pain mitigation system, the pain mitigation system may be activated before or after contact of the distal end of the device with the target tissue delivery site. For example, where the pain mitigation system is a cooling system, the pain mitigation system may be activated so that the sterile tissue contacting surface is at a desired temperature prior to contact of the sterile tissue contacting surface with the target tissue delivery site. Alternatively, where the pain mitigation system provides for pain mitigation via another mechanism, such as electrical stimulation, the pain mitigation system may be activated after contact of the sterile tissue contacting surface with the target tissue delivery site.

Following contact of the sterile tissue contacting surface with the target tissue delivery site, and actuation of a pain mitigation system if present, the active agent delivery system is actuated to deliver an amount, e.g., dosage, of an active agent to the target tissue delivery site. Where actuation of the active agent delivery system results in automated therapeutic agent delivery, the device is held in such a manner such that contact of the sterile tissue contacting surface with the target tissue delivery site is maintained during the active agent delivery. Following active agent delivery, the needle of the active agent delivery device may be withdrawn from the target tissue delivery site, e.g., using a controlled retraction profile.

The devices may be employed to deliver an active agent to a target tissue delivery site of a variety of different types of subjects. In some instances, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects are humans. The methods may be diagnostic and/or therapeutic methods.

In some instances, the methods include assembling an active agent delivery device, e.g., by operably engaging an active agent delivery system in a receiving space of an actuator component to produce a complete device, e.g., as described above. In some instances, the method includes removing the actuator component from a docking station, such as described above. In some instances, the method further includes removing the active agent delivery system from the receiving space of the actuator component. The removing may include disposing the therapeutic agent delivery system. In some instances, the method further includes docking the actuator component in the docking station.

Aspects of the invention include methods of delivery an active agent into an eye of a subject. In such embodiments, the methods may include opening a packaging, e.g., as described below, which includes a syringe pre-filled with the active agent. As reviewed above, the syringe may have a sterile interior and the active agent present therein may be sterile. The syringe may also include a non-sterile exterior surface, e.g., at least a portion of the exterior surface of the body is non-sterile, e.g., as defined above. The methods may include operably coupling a sterile needle with the syringe, e.g., by coupling a sterile needle hub to the orifice of the syringe, e.g., via a luer fitting. In certain embodiments, the non-sterile exterior surface may then be contacted to one or more surfaces, such as but not limited to, a sterile field, sterile glove, an ocular surface of the patient, a surface of the needle or hub, etc., which renders the surface non-sterile, e.g., as defined above. Following this, the active agent may be administered into the eye, e.g., by depressing the plunger of the syringe.

### Utility

Devices of the invention, e.g., as described above, find use in the delivery of a variety of different types of active agents to a target tissue delivery site to treat a variety of different types of conditions. The active agent delivery devices of the invention may be used to deliver an active agent to a variety of target tissue delivery sites. Examples of target tissue delivery sites include both external and internal delivery sites, wherein internal delivery sites include those sites located in body cavities. Examples of delivery sites include on-body and implanted drug reservoirs. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include conjunctiva, episclera, and sclera of the eye. In some instances, the subject devices are used for intravitreal injection therapy (IVT), retrobulbar injection therapy, subtenon injection therapy, subretinal injection therapy, suprachoroial injection, subconjunctival injection therapy, intracameral injection therapy, implanted reservoirs and the like.

The devices and methods of use may be employed to deliver a variety of different types of active agents. Any desired active agent composition may be delivered, where a given active agent composition may include a single active agent or combination of two or more difference active agents, as reviewed above. As reviewed above, an active agent is any component that provides pharmacological activity or other direct effect in the diagnosis, cure, mitigation, treatment, or prevention of disease, or affects the structure or any function of the body of man or animals. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids, such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; aminosterols such as squalamine; antihistamines such as H 1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; glucose modulators, e.g., insulin; immune modulators, etc.

The device may be employed to deliver a therapeutic agent to treat a variety of different disease conditions. Disease conditions of interest include, but are not limited to, ocular conditions, such as ocular disease conditions, such as intraocular neovascular disease conditions. An “intraocular neovascular disease” is a disease characterized by ocular neovascularisation. Examples of intraocular neovascular diseases include, for example, proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, Central Retinal Vein Occlusion (CRVO), Branch Retinal Vein Occlusion (BRVO), pterygium, corneal neovascularization, and retinal neovascularization. The term “age-related macular degeneration” refers to a medical condition which usually affects older adults and results in a loss of vision in the center of the visual field (the macula) because of damage to the retina. Some or all of these conditions can be treated by intravitreal injection of a VEGF-antagonist, e.g., as described above. Other ocular conditions that may be treated in accordance with aspects of the invention include, but are not limited to: retinal detachments (pneumatic retinopexy), by using devices of the invention to inject a gas into the eye, where the device may control the depth of injection to a desired/optimal depth. Disease conditions of interest also include central serous chorioretinopathy and uveitis, including anterior uveitis, pars planitis, intermediate uveitis, and posterior uveitis.

Devices of the invention, e.g., as described above, find use in the delivery of a variety of different types of compositions to subcutaneous location, e.g., for a variety of different types of applications, including aesthetic and therapeutic applications. For example, where the subcutaneous composition includes a Botulinum toxin, the methods and devices find use in aesthetic applications, e.g., elimination of wrinkles, as well as therapeutic applications, e.g., Bladder Dysfunction, Chronic Migraine, Spasticity, Cervical Dystonia, Primary Axillary Hyperhidrosis, Blepharospasm and Strabismus, etc.

### Kits

Also provided are kits that include at least one or more components, e.g., as described above. For example, a kit may include a needle and a tissue contacting tip, e.g., as described above and/or a syringe prefilled with a viscous active agent composition or a sleeve prefilled with one or more active agent solid formulations, where these components may be separate or operably engaged with each other as a composite structure. A kit may further include, where desired, an actuator component, a docking station, etc. The kit components may be present in packaging, which packaging may or may not be sterile, as desired. For example, where the kit includes a prefilled syringe, e.g., as described above, the packaging may not be exposed to sterilization following placement of the syringe in the packaging.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

Notwithstanding the appended claims, the disclosure is also defined by the following clauses:

1. An active agent delivery device, the device comprising:

(a) a syringe comprising a viscous composition of the active agent;

(b) a needle operably coupled to the syringe; and

(c) a motorized actuator configured to deliver the viscous composition from the syringe through the needle to a target delivery site.

2. The device according to Clause 1, wherein the syringe has a volume ranging from 0.1 to 5.0 ml.
3. The device according to Clause 2, wherein the syringe has a volume ranging from 0.1 to 1.50 ml.
4. The device according to Clause 3, wherein the syringe has a volume ranging from 0.1 to 1.0 ml.
5. The device according to any of the preceding clauses, wherein the device is configured to deliver a dosage having a volume ranging from 5 μl to 150 μl.
6. The device according to Clause 5, wherein the device is configured to deliver a dosage having a volume ranging from 10 μl to 50 μl or 10 μl to 100 μl.
7. The device according to any of the preceding clauses, wherein the syringe comprises a dosing mark.
8. The device according to Clause 7, wherein the dosing mark indicates delivery of a dosage that is less than the volume of the syringe.
9. The device according to Clauses 7 or 8, wherein the dosing mark ranges from 5 to 100 μL, for example where the dosing mark is 50 μL.
10. The device according to any of Clauses 1 to 6, where no dose mark is present.
11. The device according to any of the preceding clauses, wherein the syringe comprises one or more chambers.
12. The device according to any of the preceding clauses, wherein the syringe comprises a glass.
13. The device according to any of Clauses 1 to 12, wherein the syringe comprises a plastic.
14. The device according to Clause 13, wherein the plastic is selected from the group consisting of cyclic olefin polymer (COP) and cyclic olefin copolymer (COC).
15. The device according to any of the preceding clauses, wherein the syringe is not surface sterilized.
16. The device according to any of the preceding clauses, wherein the device comprises a tip comprising a sterile tissue contacting surface
17. The device according to Clause 16, wherein the sterile tissue contacting surface is planar.
18. The device according to any of Clauses 16 and 17, wherein the sterile tissue contacting surface comprises a passageway dimensioned to accommodate passage of the needle therethrough.
19. The delivery device according to any of the preceding clauses, wherein the needle has a gauge ranging from 23 to 35.
20. The delivery device according to Clause 19, wherein the needle has a gauge ranging from 23 to 30 or a gauge ranging from 30 to 33.
21. The device according to any of the preceding clauses, wherein the needle is present in a needle housing.
22. The device according to Clause 21, wherein the distal end of the syringe is operably coupled to the proximal end of the needle housing.
23. The device according to Clause 22, wherein the distal end of the syringe is operably coupled to the proximal end of the needle housing by a fitting.
24. The device according to Clause 23, wherein the fitting is a luer fitting.
25. The device according to Clause 24, wherein the luer fitting comprises a luer slip.
26. The device according to Clause 24, wherein the luer fitting comprises a luer lock.
27. The device according to any of the preceding clauses, wherein the motorized actuator delivers a force ranging 0.05 N to 250 N.
28. The device according to any of the preceding clauses, wherein the motorized actuator is configured to deliver the viscous composition in a time period ranging from 0.1 to 60 seconds.
29. The device according to Clause 28, wherein the motorized actuator is configured to deliver the viscous composition in a time period ranging from 0.1 to 60 seconds.
30. The device according to any of the preceding clauses, wherein the actuator is configured to withdraw the needle following the injection motion.
31. The device according to any of the preceding clauses, wherein the actuator component further comprises a syringe receiving space comprising the syringe.
32. The device according to any of the preceding clauses, wherein the actuator component further comprises a pain mitigation system operatively coupled to the tip.
33. The device according to Clause 32, wherein the pain mitigation system comprises an anesthesia producing system.
34. The device according to Clause 33, wherein the anesthesia producing system comprises a cooling system.
35. The device according to Clause 34, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
36. The device according to Clause 35, wherein the cooling system comprises a thermoelectric cooling system.
37. The device according to Clause 36, wherein the thermoelectric cooling system comprises a Peltier unit, a conductor coupling the Peltier unit to the tissue contacting surface and a heat sink coupled to the Peltier unit.
38. The device according to any of the preceding clauses, wherein the viscous composition has a viscosity ranging from 25 to 1500 cps.
39. The device according to Clause 38, wherein the viscous composition has a viscosity ranging from 100 to 500 cps.
40. The device according to any of the preceding clauses, wherein the liquid composition comprises a complement system modulator.
41. The device according to Clause 40, wherein the complement system modulator comprises a small molecule, antibody or binding fragment thereof.
42. The device according to any of Clauses 40 and 41, wherein the viscous composition comprises two or more active agents.
43. The device according to Clause 42, wherein the viscous composition comprises a VEGF modulator and a complement system modulator.
44. The device according to any of the preceding clauses, wherein the device is a handheld device.
45. The device according to any of the preceding clauses, wherein the target delivery site is an ocular delivery site.
46. The device according to Clause 45, wherein the ocular deliver site is an intravitreal delivery site.
47. A syringe that is not surface sterilized and comprises a sterile viscous composition comprising an active agent.
48. The syringe according to Clause 47, wherein the syringe has a volume ranging from 0.1 to 5.0 ml.
49. The syringe according to Clause 48, wherein the syringe has a volume ranging from 0.1 to 1.50 ml.
50. The syringe according to Clause 49, wherein the syringe has a volume ranging from 0.1 to 1.0 ml.
51. The syringe according to any of Clauses 47 to 50, wherein the syringe is configured to deliver a dosage having a volume ranging from 5 μl to 150 μl.
52. The syringe according to Clause 51, wherein the syringe is configured to deliver a dosage having a volume ranging from 10 μl to 50 μl.
53. The syringe according to any of Clauses 47 to 52, wherein the syringe comprises a dosing mark.
54. The syringe according to Clause 53, wherein the dosing mark indicates delivery of a dosage that is less than the volume of the syringe.
55. The syringe according to Clauses 53 or 54, wherein the dosing mark ranges from 10 to 100 μL, such as where the dosing mark is 50 μL.
56. The syringe according to any of Clauses 47 to 52, wherein the syringe does not include a dosing mark.
57. The syringe according to any of Clauses 47 to 56, wherein the viscous composition has a viscosity ranging from 25 to 1500 cps.
58. The syringe according to Clause 57, wherein the viscous composition has a viscosity ranging from 100 to 500 cps.
59. The syringe according to any of Clauses 47 to 58, wherein the viscous composition comprises a complement system modulator.
60. The syringe according to Clause 59, wherein the complement system modulator comprises a small molecule, antibody or binding fragment thereof.
61. The syringe according to Clause 59, wherein the viscous composition comprises two or more active agents.
62. The syringe according to Clause 60, wherein the viscous composition comprises two or more active agents.
63. The syringe according to Clause 62, wherein the viscous composition comprises a VEGF modulator and a complement system modulator.
64. The syringe according to any of Clauses 47 to 63, wherein the syringe comprises a distal end removable cap.
65. The syringe according to Clause 64, wherein removal of the removable cap exposes a fitting.
66. The syringe according to Clause 65, wherein the fitting comprises a luer fitting.
67. The syringe according to Clause 66, wherein the luer fitting comprises a luer slip.
68. The syringe according to Clause 66, wherein the luer fitting comprises a luer lock.
69. The syringe according to any of Clauses 47 to 68, wherein the syringe comprises a glass.
70. The syringe according to any of Clauses 47 to 68, wherein the syringe comprises a plastic.
71. The syringe according to Clause 70, wherein the plastic is selected from the group consisting of cyclic olefin polymer (COP) and cyclic olefin copolymer (COC).
72. An actuator component for an active agent delivery device comprising a syringe comprising a viscous composition of the active agent and a needle operably coupled to the syringe, the actuator component comprising:

a motorized actuator configured to move the viscous composition through the needle.

73. The actuator component according to Clause 72, wherein the motorized actuator delivers a force ranging 0.05 N to 250 N.
74. The actuator component according to any of Clauses 72 to 73, wherein the motorized actuator is configured to deliver the viscous composition in a time period ranging from 0.1 to 60 seconds.
75. The actuator component according to Clause 74, wherein the motorized actuator is configured to deliver the viscous composition in a time period ranging from 0.1 to 60 seconds.
76. The actuator component according to any of Clauses 72 to 74, wherein the actuator is configured to sequentially perform a first priming motion and a second injection motion.
77. The actuator component according to Clause 76, wherein the actuator is further configured to withdraw the needle following the second injection motion.
78. The actuator component according to any of Clauses 72 to 77, wherein the actuator component further comprises a syringe receiving space configured to receive the syringe.
79. The actuator component according to any of Clauses 72 to 78, wherein the actuator component further comprises a pain mitigation system operatively coupled to the tip.
80. The actuator component according to Clause 79, wherein the pain mitigation system comprises an anesthesia producing system.
81. The actuator component according to Clause 80, wherein the anesthesia producing system comprises a cooling system.
82. The actuator according to Clause 81, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
83. The actuator according to Clause 82, wherein the cooling system comprises a thermoelectric cooling system.
84. The actuator according to Clause 83, wherein the thermoelectric cooling system comprises a Peltier unit, a conductor coupling the Peltier unit to the tissue contacting surface and a heat sink coupled to the Peltier unit.
85. The actuator according to Clause 82, wherein the cooling system comprises a low-temperature substance cooling system.
86. A method of delivering an active agent to an ocular target delivery site, the method comprising:

(a) contacting a distal end of an active agent delivery device with a topical ocular location, the active agent delivery device comprising:
(i) a syringe comprising a viscous composition of the active agent;
(ii) a needle operably coupled to the syringe; and
(iii) a motorized actuator configured to deliver the viscous composition from the syringe through the needle to the ocular target delivery site; and

(b) actuating the actuator to move the viscous composition from the syringe through the needle so as to deliver the active agent to the ocular target delivery site.

87. The method according to Clause 86, wherein actuating comprises moving the needle.
88. The method according to any of Clauses 86 and 87, wherein method further comprises assembling the device.
89. The method according to Clause 88, wherein assembling the device comprises operably coupling the syringe with a syringe receiving space of the actuator.
90. The method according to any of Clauses 86 to 89, wherein actuator further comprises a pain mitigation system and the method further comprises actuating a pain mitigation system.
91. The method according to any of Clauses 86 to 90, wherein the viscous composition has a viscosity ranging from 25 to 1500 cps.
92. The method according to Clause 91, wherein the viscous composition has a viscosity ranging from 100 to 500 cps.
93. The method according to any of Clauses 86 to 92, wherein the viscous composition comprises a complement system modulator.
94. The method according to Clause 93, wherein the complement system modulator comprises a small molecule, antibody or binding fragment thereof.
95. The method according to any of Clauses 93 and 94, wherein the viscous composition comprises two or more active agents.
96. The method according to Clause 95, wherein the viscous composition comprises a VEGF modulator and a complement system modulator.
97. The method according to any of Clauses 86 to 96, wherein the topical ocular location is positioned within 0.5 mm to 4 mm of the limbus.
98. The method according to any of Clauses 86 to 97, wherein the ocular target delivery site is an intravitreal site.
99. The method according to any of Clauses 86 to 98, wherein the ocular target delivery site is an intracameral site.
100. The method according to any of Clauses 86 to 99, wherein the method is a method of treating a subject for an ocular disease.
101. A kit comprising:

(a) a needle; and

(b) a syringe comprising a viscous composition comprising an active agent.

102. The kit according to Clause 101, wherein the viscous composition has a viscosity ranging from 25 to 1500 cps.
103. The kit according to Clause 102, wherein the viscous composition has a viscosity ranging from 100 to 500 cps.
104. The kit according to any of Clauses 101 to 103, wherein the viscous composition comprises a complement system modulator.
105. The kit according to Clause 104, wherein the complement system modulator comprises a small molecule, antibody or binding fragment thereof.
106. The kit according to any of Clauses 104 and 105, wherein the viscous composition comprises two or more active agents.
107. The kit according to Clause 106, wherein the viscous composition comprises a VEGF modulator and a complement system modulator.
108. The kit according to any of Clauses 101 to 107, wherein the syringe is not surface sterilized.
109. The kit according to Clause 108, wherein the kit comprises an active agent delivery device tip component comprising:
(i) a tip comprising a sterile tissue contacting surface; and
(ii) the needle operably coupled to the tip;

wherein the tip component is present in a sealed housing comprising a sterile interior and non-sterile exterior.

110. The kit according to Clause 109, wherein the sterile tissue contacting surface comprises a passageway dimensioned to accommodate passage of the needle therethrough.
111. The kit according to Clause 110, wherein the distal end of the needle is movable relative to the sterile tissue contacting surface.
112. The kit according to any Clauses 101 to 111, wherein the needle has a gauge ranging from 23 to 35.
113. The kit according to any of Clauses 101 to 112, wherein the syringe has a volume ranging from 0.1 to 5.0 ml.
114. The kit according to Clause 113, wherein the syringe has a volume ranging from 0.1 to 1.50 ml.
115. The kit according to Clause 114, wherein the syringe has a volume ranging from 0.1 to 1.0 ml.
116. The kit according to any of Clauses 101 to 115, wherein the syringe is configured to deliver a dosage having a volume ranging from 5 μl to 150 μl.
117. An active agent delivery device, the device comprising:

(a) a sleeve comprising a solid formulation of the active agent;

(b) a needle operably coupled to the sleeve; and

(c) an actuator configured to move the solid formulation from the sleeve and through the needle;

wherein the device is configured to deliver the solid formulation to an ocular location.

118. The delivery device according to Clause 117, wherein the solid formulation comprises the active agent in a solid matrix.
119. The delivery device according to Clause 118, wherein the solid matrix comprises a polymeric matrix.
120. The delivery device according to Clause 119, wherein the polymeric matrix comprises a biodegradable polymer.
121. The delivery device according to Clause 120, wherein the biodegradable polymer is selected from the group consisting of polylactides, poly(lactide-co-glycolides), polycaprolactones, and combinations thereof.
122. The delivery device according to any of Clauses 117 to 121, wherein the solid formulation has a longest dimension ranging from 0.5 to 30,000 μm.
123. The delivery device according to any of Clauses 117 to 122, wherein the solid formulation comprises a rod-shaped configuration.
124. The delivery device according to any of Clauses 117 to 123, wherein the active agent comprises a steroid.
125. The delivery device according to Clause 124, wherein the steroid is selected from the group consisting of cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof.
126. The delivery device according to any of Clauses 117 to 125, wherein the active agent comprises an intraocular pressure lowering agent.
127. The delivery device according to Clause 126, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, antibody, hydrogel, and combinations thereof.
128. The delivery device according to any of Clauses 117 to 127, wherein the sleeve comprises a single solid formulation.
129. The delivery device according to any of Clauses 117 to 127, wherein the sleeve comprises multiple solid formulations.
130. The delivery device according to Clause 129, wherein the sleeve comprises 2 to 10 solid formulations.
131. The delivery device according to any of Clauses 117 to 130, wherein the needle has a gauge ranging from 23 to 35.
132. The delivery device according to any of Clauses 117 to 131, wherein the distal end of the sleeve is operably coupled to the proximal end of the needle housing by a fitting.
133. The delivery device according to Clause 132, wherein the fitting is a luer fitting.
134. The delivery device according to Clause 133, wherein the luer fitting comprises a luer slip or luer lock.
135. The delivery device according to Clause 133, wherein the the sleeve is operably coupled to the needle as a single piece.
136. The delivery device according to any of Clauses 117 to 135, wherein the actuator comprises a motor.
137. The delivery device according to any of Clauses 117 to 135, wherein the actuator comprises a spring.
138. The delivery device according to any of Clauses 117 to 135, wherein the actuator comprises a manual actuator.
139. The delivery device according to any of Clauses 117 to 138, wherein the actuator is further configured to withdraw the needle following the second injection motion.
140. The delivery device according to any of Clauses 117 to 139, wherein the actuator component further comprises a sleeve receiving space comprising the sleeve.
141. The delivery device according to any of Clauses 117 to 140, wherein the actuator component further comprises a pain mitigation system.
142. The delivery device according to Clause 141, wherein the pain mitigation system comprises an anesthesia producing system.
143. The delivery device according to Clause 142, wherein the anesthesia producing system comprises a cooling system.
144. The delivery device according to Clause 143, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
145. The delivery device according to Clause 144, wherein the cooling system comprises a thermoelectric cooling system.
146. The delivery device according to Clause 145, wherein the thermoelectric cooling system comprises a Peltier unit, a conductor coupling the Peltier unit to the tissue contacting surface and a heat sink coupled to the Peltier unit.
147. The delivery device according to any of Clauses 117 to 146, wherein the device is a handheld device.
148. The delivery device according to any of Clauses 117 to 147, wherein the ocular location is an intravitreal location.
149. The delivery device according to any of Clauses 117 to 148, wherein the ocular location is an intracameral location.
150. The delivery device according to any of Clauses 117 to 149, wherein the device further comprises a distal end tip.
151. The delivery device according to Clause 150, wherein the distal end tip is configured to collapse towards the proximal end of the device during use.
152. The delivery device according to Clause 151, wherein the distal end tip is configured to collapse towards the proximal end of the device by a distance ranging from 2 to 10 mm.
153. The delivery device according to any of Clauses 151 to 152, wherein collapse of the distal end tip exposes the needle.
154. A sleeve for use with an ocular delivery device and comprising a solid formulation of an active agent.
155. The sleeve according to Clause 154, wherein the solid formulation comprises the active agent in a solid matrix.
156. The sleeve according to Clause 155, wherein the solid matrix comprises a polymeric matrix.
157. The sleeve according to Clause 156, wherein the polymeric matrix comprises a biodegradable polymer.
158. The sleeve according to Clause 157, wherein the biodegradable polymer is selected from the group consisting of polylactides, poly(lactide-co-glycolides), polycaprolactones, and combinations thereof.
159. The sleeve according to any of Clauses 154 to 158, wherein the solid formulation has a longest dimension ranging from 0.5 to 30,000 μm.
160. The sleeve according to any of Clauses 154 to 159, wherein the solid formulation comprises a rod-shaped configuration.
161. The sleeve according to any of Clauses 154 to 160, wherein the active agent comprises a steroid.
162. The sleeve according to Clause 161, wherein the steroid is selected from the group consisting of cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof.
163. The sleeve according to any of Clauses 154 to 162, wherein the active agent comprises an intraocular pressure lowering agent or a neuroprotective agent.
164. The sleeve according to Clause 163, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, anti-VEGF agent, hydrogels, and combinations thereof.
165. The sleeve according to any of Clauses 154 to 164, wherein the sleeve comprises a single solid formulation.
166. The sleeve according to any of Clauses 154 to 164, wherein the sleeve comprises multiple solid formulations.
167. The sleeve according to Clause 166, wherein the sleeve comprises 2 to 10 solid formulations.
168. The sleeve according to any of Clauses 154 to 167, wherein the sleeve comprises a plastic.
169. The sleeve according to Clause 168, wherein the plastic is selected from the group consisting of cyclic olefin polymer (COP) and cyclic olefin copolymer (COC).
170. The sleeve according to any of Clauses 154 to 167, wherein the sleeve comprises a glass.
171. The sleeve according to any of Clauses 154 to 170, wherein the sleeve is not surface sterilized.
172. The sleeve according to any of Clauses 154 to 171, wherein the sleeve is surface sterilized.
173. The sleeve according to any of Clauses 154 to 172, wherein the sleeve has one or more removable barriers
174. The sleeve according to Clause 173, wherein the one or more removable barriers can be manually removed
175. The sleeve according to Clause 173 to 174, wherein the removable barrier can be punctured by the actuator.
176. The sleeve according to Clause 175, wherein the removable barrier can be punctured by the actuator driving the solid formulation through the barrier.
177. An actuator component for an active agent delivery device comprising a sleeve comprising a solid formulation, the actuator component comprising:

an actuator configured to move the solid formulation from the sleeve and through a needle.

178. The actuator component according to Clause 177, wherein the actuator comprises a motor.
179 The actuator component according to Clause 177, wherein the actuator comprises a spring.
180. The actuator component according to Clause 177, wherein the actuator comprises a manual actuator.
181. The actuator component according to any of Clauses 177 to 180, wherein the actuator is further configured to withdraw the needle following administration of the solid formulation.
182. The actuator component according to any of Clauses 177 to 181, wherein the actuator component comprises a sleeve receiving space configured to receive the sleeve.
183. The actuator component according to any of Clauses 177 to 182, wherein the actuator component further comprises a pain mitigation system operatively coupled to the tip.
184. The actuator component according to Clause 183, wherein the pain mitigation system comprises an anesthesia producing system.
185. The actuator component according to Clause 184, wherein the anesthesia producing system comprises a cooling system.
186. The actuator according to Clause 185, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
187. The actuator according to Clause 186, wherein the cooling system comprises a thermoelectric cooling system.
188. The actuator according to Clause 187, wherein the thermoelectric cooling system comprises a Peltier unit, a conductor coupling the Peltier unit to the tissue contacting surface and a heat sink coupled to the Peltier unit.
189. The actuator component according to any of Clauses 177 to 188, wherein the actuator component is present in a docking station.
190. A method of delivering an active agent to an intravitreal location, the method comprising:

(a) contacting a distal end of an active agent delivery device with a topical ocular location, the active agent delivery device comprising:
(i) a sleeve comprising a solid formulation of the active agent;
(ii) a needle operably coupled to the sleeve; and
(iii) an actuator configured to move the solid formulation from the sleeve and through the needle; and

(b) actuating the actuator to move the solid formulation from the sleeve through the needle so as to deliver the active agent to the intravitreal location.

191. The method according to Clause 190, wherein actuating comprises moving the needle.
192. The method according to Clause 191, wherein actuating comprises moving the needle outside a collapsible tip by collapsing the collapsible tip.
193. The method according to any of Clauses 190 to 192, wherein method further comprising assembling the device.
194. The method according to Clause 192, wherein assembling the device comprises operably coupling the sleeve with a sleeve receiving space of the actuator.
195. The method according to any of Clauses 190 to 194, wherein actuator further comprises a pain mitigation system and the method further comprises actuating a pain mitigation system.
196. The method according to any of Clauses 190 to 195, wherein the solid formulation comprises the active agent in a solid matrix.
197. The method according to Clause 196, wherein the solid matrix comprises a polymeric matrix.
198. The method according to Clause 197, wherein the polymeric matrix comprises a biodegradable polymer.
199. The method according to Clause 198, wherein the biodegradable polymer is selected from the group consisting of polylactides, poly(lactide-co-glycolides), polycaprolactones, and combinations thereof.
200. The method according to any of Clauses 190 to 199, wherein the solid formulation comprises a rod-shaped configuration.
201. The method according to any of Clauses 190 to 200, wherein the active agent comprises a steroid.
202. The method according to Clause 201, wherein the steroid is selected from the group consisting of cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof.
203. The method according to any of Clauses 190 to 202, wherein the active agent comprises an intraocular pressure lowering agent or a neuroprotective agent.
204. The method according to Clause 203, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, anti-VEGF agent, hydrogel, and combinations thereof.
205. The method according to any of Clauses 190 to 204, wherein the sleeve comprises a single solid formulation.
206. The method according to any of Clauses 190 to 204, wherein the sleeve comprises multiple solid formulations.
207. The method according to Clause 206, wherein the sleeve comprises 2 to 10 solid formulations.
208. The method according to any of Clauses 190 to 207, wherein the topical ocular location is positioned within 0.5 mm to 4 mm of the limbus.
209. The method according to any of Clauses 190 to 208, wherein the method is a method of treating a subject for an ocular disease.
210. A kit comprising:

(a) a sleeve comprising a solid formulation of an active agent; and

(b) a needle configured to be operably coupled to the sleeve.

211. The kit according to Clause 210, wherein the solid formulation comprises the active agent in a solid matrix.
212. The kit according to Clause 211, wherein the solid matrix comprises a polymeric matrix.
213. The kit according to Clause 212, wherein the polymeric matrix comprises a biodegradable polymer.
214. The kit according to Clause 213, wherein the biodegradable polymer is selected from the group consisting of polylactides, poly(lactide-co-glycolides), polycaprolactones, and combinations thereof.
215. The kit according to any of Clauses 210 to 214, wherein the solid formulation has a longest dimension ranging from 0.5 to 30,000 μm.
216. The kit according to any of Clauses 210 to 215, wherein the solid formulation comprises a rod-shaped configuration.
217. The kit according to any of Clauses 210 to 216, wherein the active agent comprises a steroid.
218. The kit according to Clause 217, wherein the steroid is selected from the group consisting of cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof.
219. The kit according to any of Clauses 210 to 218, wherein the active agent comprises an intraocular pressure lowering agent or a neuroprotective agent.
220. The kit according to Clause 219, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, anti-VEGF agent, hydrogels, and combinations thereof.
221. The kit according to any of Clauses 210 to 220, wherein the sleeve comprises a single solid formulation.
222. The kit according to any of Clauses 210 to 221, wherein the sleeve comprises multiple solid formulations.
223. The kit according to Clause 222, wherein the sleeve comprises 2 to 10 solid formulations.
224. The kit according to any of Clauses 210 to 223, wherein the sleeve has one more removable barriers
225. The kit according to any of Clauses 210 to 224, wherein the needle has a gauge ranging from 23 to 35.
226. The kit according to any of Clauses 210 to 225, wherein the kit further comprises a tip that contains the needle and that can be operably coupled to the sleeve.
227. A subcutaneous delivery device, the device comprising:

(a) a syringe comprising a composition for subcutaneous delivery;

(b) a needle operably coupled to the syringe;

(c) a motorized actuator configured to deliver the composition from the syringe through the needle to a subcutaneous location; and

(d) a pain mitigation system.

228. The device according to Clause 227, wherein the syringe has a volume ranging from 0.1 to 5.0 ml.
229. The device according to Clause 228, wherein the device is configured to deliver a dosage having a volume ranging from 5 μl to 3500 μl.
230. The device according to any of Clauses 227 to 229, wherein the syringe comprises a dosing mark.
231. The device according to any of Clauses 227 to 229, wherein the syringe does not include a dosing mark.
232. The device according to any of Clauses 227 to 231, wherein the syringe is a commercially available, off-the-shelf syringe
233. The device according to any of Clauses 227 to 232, wherein the syringe comprises one or more chambers.
234. The device according to any of Clauses 227 to 233, wherein the syringe comprises a glass.
235. The device according to any of Clauses 227 to 233, wherein the syringe comprises a plastic.
236. The device according to Clause 235, wherein the plastic is selected from the group consisting of cyclic olefin polymer (COP) and cyclic olefin copolymer (COC).
237. The device according to any of Clauses 227 to 236, wherein the syringe is not surface sterilized.
238. The device according to any of Clauses 227 to 237, wherein the device comprises a tip comprising a sterile tissue contacting surface, preferably, wherein the sterile tissue contacting surface has an area ranging from 0.25 to 1 mm², such as 0.5 mm².
239. The device according to any of Clauses 237 to 238, wherein the sterile tissue contacting surface is planar.
240. The device according to any of Clauses 237 to 239 wherein the sterile tissue contacting surface comprises a passageway dimensioned to accommodate passage of the needle therethrough.
241. The delivery device according to any of Clauses 227 to 240, wherein the needle has a gauge ranging from 23 to 35.
242. The device according to any of Clauses 227 to 241, wherein the needle is present in a needle housing.
243. The device according to Clause 242, wherein the distal end of the syringe is operably coupled to the proximal end of the needle housing.
244. The device according to Clause 243, wherein the distal end of the syringe is operably coupled to the proximal end of the needle housing by a fitting, e.g., such as a luer fitting, e.g., a luer slip or luer lock.
245. The device according to any of Clauses 227 to 244, wherein the motorized actuator delivers a force ranging 0.05 N to 250 N.
246. The device according to any of Clauses 227 to 245, wherein the motorized actuator is configured to deliver the composition in a time period ranging from 0.1 to 60 seconds.
247. The device according to any of Clauses 227 to 246, wherein the actuator is configured to withdraw the needle following the injection motion.
248. The device according to any of Clauses 227 to 247, wherein the actuator component further comprises a syringe receiving space comprising the syringe.
249. The device according to any of Clauses 227 to 248, wherein the pain mitigation system comprises an anesthesia producing system.
250. The device according to Clause 249, wherein the anesthesia producing system comprises a cooling system.
251. The device according to Clause 250, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system, and preferably comprises a thermoelectric cooling system.
252. The device according to Clause 251, wherein the thermoelectric cooling system comprises a Peltier unit, a conductor coupling the Peltier unit to the tissue contacting surface and a heat sink coupled to the Peltier unit.
253. The device according to any of Clauses 227 to 252, wherein the composition is an aesthetic composition.
254. The device according to Clause 253, wherein the aesthetic composition comprises a Botulinum toxin.
255. The device according to Clause 253, wherein the aesthetic composition comprises a dermal filler.
256. The device according to any of Clauses 227 to 252, wherein the composition comprises an immune system modulator
257. The device according to any of Clauses 227 to 252, wherein the composition comprises a glucose modulator.
258. The device according to any of Clauses 227 to 257, wherein the device is a handheld device.
259. The device according to any of Clauses 227 to 258, wherein the target subcutaneous delivery site is maxilofacial site.
260. A syringe that is not surface sterilized and comprises aesthetic composition for subcutaneous delivery.
261. The syringe according to Clause 260, wherein the syringe has a volume ranging from 0.1 to 5.0 ml.
262. The syringe according to any of Clauses 260 to 261, wherein the syringe is configured to deliver a dosage having a volume ranging from 5 μl to 3500 μl.
263. The syringe according to any of Clauses 260 to 262, wherein the syringe comprises a dosing mark.
264. The syringe according to any of Clauses 260 to 262, wherein the syringe does not include a dosing mark.
265. The syringe according to any of Clauses 260 to 264, wherein the aesthetic composition comprises a dermal filler or botulinum toxin.
266. The syringe according to any of Clauses 260 to 265, wherein the syringe comprises a distal end removable cap.
267. The syringe according to Clause 266, wherein removal of the removable cap exposes a fitting.
268. The syringe according to Clause 267, wherein the fitting comprises a luer fitting.
269. The syringe according to Clause 268, wherein the luer fitting comprises a luer slip.
270. The syringe according to Clause 269, wherein the luer fitting comprises a luer lock.
271. The syringe according to any of Clauses 260 to 270, wherein the syringe comprises a glass.
272. The syringe according to any of Clauses 260 to 270, wherein the syringe comprises a plastic.
273. The syringe according to Clause 272, wherein the plastic is selected from the group consisting of cyclic olefin polymer (COP) and cyclic olefin copolymer (COC).
274. A syringe that is not surface sterilized and comprises an immune modulator for subcutaneous delivery.
275. A syringe that is terminally surface sterilized and comprises an immune modulator for subcutaneous delivery.

276. A syringe that is not surface sterilized and comprises a glucose modulator for subcutaneous delivery.

277. A syringe that is terminally surface sterilized and comprises a glucose modulator for subcutaneous delivery.
278. A method of delivering a composition to a subcutaneous location, the method comprising:

(a) contacting a distal end of a subcutaneous delivery device with a topical dermal location, the active agent delivery device comprising:
(i) a syringe comprising a composition for subcutaneous delivery;
(ii) a needle operably coupled to the syringe;
(iii) a motorized actuator configured to deliver the composition from the syringe through the needle to a subcutaneous target delivery site; and
(iv) a pain mitigation system; and

(b) actuating the actuator to move the composition from the syringe through the needle so as to deliver the active agent to the subcutaneous location.

279. The method according to Clause 278, wherein actuating comprises moving the needle.
280. The method according to any of Clauses 278 and 279, wherein method further comprises assembling the device.
281. The method according to Clause 280, wherein assembling the device comprises operably coupling the syringe with a syringe receiving space of the actuator.
282. The method according to any of Clauses 278 to 281, wherein the composition is an aesthetic composition.
283. The method according to Clause 282, wherein the aesthetic composition comprises a Botulinum toxin.
284. The method according to Clause 282, wherein the aesthetic composition comprises a dermal filler.
285. The method according to any of Clauses 278 to 281, wherein the composition comprises an immune modulator.
286. The method according to any of Clauses 278 to 281, wherein the composition comprises a glucose modulator.
287. The method according to any of Clauses 278 to 286, wherein the subcutaneous location is a maxillofacial, i.e., head/neck, location.
288. The method according to any of Clauses 278 to 286, wherein the subcutaneous location is the bladder.
289. The method according to any of Clauses 278 to 286, wherein the subcutaneous location is the axillary region.
290. The method according to any of Clauses 278 to 286, wherein the subcutaneous location is a thigh region.
291. The method according to any of Clauses 278 to 286, wherein the subcutaneous location is an abdominal region
292. A kit comprising:

(a) a needle; and

(b) a tip comprising a sterile tissue contacting surface, wherein the tip is configured to be operably coupled to the needle.

293. The kit according to Clause 292, wherein the kit further comprises a syringe.
294. The kit according to Clause 293, wherein the syringe is not surface sterilized.
295. The kit according Clause 294, wherein the syringe is surface sterilized.
296. The kit according to Clauses 293 to 295, wherein the syringe comprises a composition for subcutaneous delivery.
297. The kit according to Clause 296, wherein the composition is an aesthetic composition.
298. The kit according to Clause 297, wherein the composition is a dermal filler.
299. The kit according to any of Clause 296, wherein the composition comprises an immune modulator.
300. The kit according to Clause 296, wherein the kit comprises a glucose modulator.
301. The kit according to any of Clauses 293 to 300, wherein the syringe has a volume ranging from 0.1 to 5.0 ml.
302. The kit according to any of Clauses 293 to 300, wherein the syringe is configured to deliver a dosage having a volume ranging from 5 μl to 3500 μl.
303. The kit according to any of Clauses 293 to 302, wherein the syringe comprises a dosing mark.
304. The kit according to any of Clauses 292 to 303, wherein the tip component is present in a sealed housing comprising a sterile interior and non-sterile exterior.
305. The kit according to Clause 304, wherein the sterile tissue contacting surface comprises a passageway dimensioned to accommodate passage of the needle therethrough.
306. The kit according to Clause 305, wherein the distal end of the needle is movable relative to the sterile tissue contacting surface.
307. The kit according to any of Clauses 292 to 306, wherein the needle has a gauge ranging from 23 to 35.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. An active agent delivery device, wherein the device is either:
(a) a device configured for delivery of a viscous composition, wherein the device comprises:
(i) a syringe comprising a viscous composition of the active agent;
(ii) a needle operably coupled to the syringe; and
(iii) a motorized actuator configured to deliver the viscous composition from the syringe through the needle to a target delivery site;
(b) a device configured to deliver a solid formulation to an ocular location, wherein the device comprises:
(i) a sleeve comprising a solid formulation of the active agent;
(ii) a needle operably coupled to the sleeve; and
(iii) an actuator configured to move the solid formulation from the sleeve and through the needle; or
(c) a subcutaneous delivery device, wherein the device comprises:
(i) a syringe comprising a composition for subcutaneous delivery;
(ii) a needle operably coupled to the syringe;
(iii) a motorized actuator configured to deliver the composition from the syringe through the needle to a subcutaneous location; and
(iii) a pain mitigation system.

2. The device according to claim 1, wherein the device is configured for delivery of a viscous composition.

3. The device according to claim 2, wherein the syringe has a volume ranging from 0.1 to 5.0 ml.

4. The device according to claim 3, wherein the device is configured to deliver a dosage having a volume ranging from 5 μl to 500 μl.

5. The device according to any of claims 2 to 4, wherein the motorized actuator delivers a force ranging 0.05 N to 250 N.

6. The device according to any of claims 2 to 5, wherein the motorized actuator is configured to deliver the viscous composition in a time period ranging from 0.1 to 60 seconds.

7. The device according to any of claims 2 to 6, wherein the viscous composition has a viscosity ranging from 25 to 1500 cps.

8. The device according to claim 1, wherein device configured to deliver a solid formulation to an ocular location.

9. The delivery device according to claim 8, wherein the solid formulation comprises the active agent in a solid matrix.

10. The delivery device according to claim 9, wherein the solid matrix comprises a polymeric matrix.

11. The delivery device according to claim 1, wherein the delivery device is a subcutaneous delivery device.

12. The device according to claim 11, wherein the composition is an aesthetic composition.

13. The device according to claim 12, wherein the aesthetic composition comprises a Botulinum toxin.

14. The device according to claim 12, wherein the device is configured to deliver a dosage volume of 0.1 ml multiple times from the same syringe.

15. A method comprising delivery a composition using a device according to any of claims 1 to 14.
